(19) United States
(12) Patent Application Publication
(10) Pub. No.: US 2023/0337699 A1
(43) Pub. Date: Oct. 26, 2023

(54) RIBULOSE-1,5-BISPHOSPHATE CARBOXYLATE-OXYGENASE (RUBISCO) PROTEIN ISOLATE COMPOSITIONS FOR PLANT-BASED EGG REPLACEMENT PRODUCTS AND PLANT-BASED MILK REPLACEMENT PRODUCTS

(71) Applicant: Plantible Foods Inc., San Marcos, CA (US)

(72) Inventors: Parker LEE, Poway, CA (US); Tony MARTENS FEKINI, Encinitas, CA (US); Clara RACETTE, San Diego, CA (US)

(21) Appl. No.: 18/121,210

(22) Filed: Mar. 14, 2023

Related U.S. Application Data
(63) Continuation of application No. PCT/US22/25737, filed on Apr. 21, 2022.
(60) Provisional application No. 63/289,650, filed on Dec. 15, 2021.
(60) Provisional application No. 63/208,151, filed on Jun. 8, 2021.
(60) Provisional application No. 63/178,657, filed on Apr. 23, 2021.

(51) Int. Cl. A23J 3/14; C07K 14/415; A23L 29/269; A23L 29/30

(52) U.S. Cl. CPC A23J 3/14; C07K 14/415; A23L 29/269; A23L 29/37

(57) ABSTRACT

Provided are compositions of high purity plant derived proteins for use, optionally, in plant-based food products. In particular, protein isolates described includes a RuBisCO protein isolate, optionally for combination with a food component and/or a food additive. The food additive includes: a plasticizer, an oil, a sugar, a flavoring component, a coloring component, a fiber, a soluble salt, a starch, an acid, and/or a wax. The plant-based food product is a milk replacement product or an egg replacement product.

### CROSS-REFERENCE

This application is a continuation application of U.S. PCT Application No. PCT/US2022/025737, filed Apr. 21, 2022, which claims benefit of U.S. Provisional Application No. 63/178,657, filed on Apr. 23, 2021; 63/208,151 filed on Jun. 8, 2021; and 63/289,650 filed on Dec. 15, 2021, each of which are incorporated herein by reference in its entirety.

### SEQUENCE LISTING

The instant application contains a Sequence Listing which has been submitted electronically in ASCII format and is hereby incorporated by reference in its entirety. Said ASCII copy, created on May 23, 2022, is named 200834_704301_SL.txt and is 28,857 bytes in size.

### BACKGROUND

Animal-based sources of protein are known to be associated with environmental risk from their caretaking and health risks to consumers, among other concerns. Thus, there is a need for alternative sources of protein.

Plant-based food products provide numerous benefits as compared to the food products they replace. For example, plant-based food products provide health benefits (e.g., less cholesterol or lower levels of saturated fats) and eliminate the negative aspects of animal husbandry, including the environmental impacts of such, the animal confinement, the disruption of maternal-offspring interactions, and the slaughter of animals for their meat.

An example of a plant-based food product comprises an egg replacement product. However, such product requires the addition of emulsifiers, relies on ingredients that are not considered “clean-label,” and/or is low in protein.

Another example of a plant-based food product comprises a milk replacement product. However, such product relies on ingredients that are not considered “clean-label” and/or is low in protein.

Thus, there is a need for animal replacement protein sources for egg and milk or egg- and milk-based products providing higher protein than commercially available plant-based protein sources.

### BRIEF SUMMARY

Provide herein are compositions, wherein the compositions comprise: a ribulose-1,5-bisphosphate carboxylase/oxygenase (RuBisCO) protein isolate; an aqueous solution; and a plasticizer, wherein a ratio by weight of the plasticizer to the RuBisCO protein isolate is about 1:5 to 1:200, or wherein the plasticizer is present in amount of up to about 8% by weight.

Provided herein are compositions where the compositions comprise: a ribulose-1,5-bisphosphate carboxylase/oxygenase (RuBisCO) protein isolate, wherein the RuBisCO protein isolate is from a floating aquatic plant; and an aqueous solution, wherein a ratio by weight of the RuBisCO protein isolate to the aqueous solution is 1:4 to 1:100, or wherein the aqueous solution is present in an amount of at least 50% by weight.

Provided herein are compositions wherein the compositions comprise: a ribulose-1,5-bisphosphate carboxylase/oxygenase (RuBisCO) protein isolate; and an oil; wherein a ratio by weight of the RuBisCO protein isolate to the oil is 10:1 to 1:20; or wherein the oil is present in amount of up to about 20% by weight.

Provided herein are compositions, wherein the compositions comprise: a ribulose-1,5-bisphosphate carboxylase/oxygenase (RuBisCO) protein isolate, wherein the RuBisCO protein isolate is present in an amount of at least 70% by weight of the composition; a lipid; and a thickener, wherein the composition is in the form of a powder.

Provided herein are methods of manufacturing, comprising: spray drying or freeze drying the composition described herein. Provided herein are methods of manufacturing, comprising: providing a ribulose-1,5-bisphosphate carboxylase/oxygenase (RuBisCO) protein isolate, and mixing the RuBisCO with at least one of the following: an oil; wherein a ratio by weight of the RuBisCO protein isolate to the oil is 10:1 to 1:20; or wherein the oil is present in amount of up to about 20% by weight; a plasticizer, wherein a ratio by weight of the plasticizer to the RuBisCO protein isolate is about 1:5 to 1:200, or wherein the plasticizer is present in amount of up to about 8% by weight; and an aqueous solution, wherein a ratio by weight of the RuBisCO protein isolate to the aqueous solution is 1:4 to 1:100, or wherein the aqueous solution is present in an amount of at least 50% by weight.

### BRIEF DESCRIPTION OF THE DRAWINGS

FIG. 1 depicts a block diagram of a plant-based food product.

FIG. 2 and FIG. 3 depict graphical plots showing gelation rheology results and a modulus comparison of RuBisCO protein isolate samples as compared to an egg white protein (EWP) sample.

FIG. 4 depicts a graphical plot of an amino acid profile of a RuBisCO protein isolate and an amino acid profile of a whole egg.

FIG. 5 depicts a graphical plot of essential amino acids for a dietary reference, a RuBisCO protein isolate, and a whole egg.

FIG. 6 depicts a graphical plot of an oil holding capacity of a RuBisCO protein isolate and an oil holding capacity of an egg white.

FIG. 7 depicts a graphical plot of an emulsion activity of a RuBisCO protein isolate and an emulsion activity of an egg white.

FIG. 8 depicts a graphical plot of a foam capacity of a RuBisCO protein isolate and a foam capacity of an egg white.

FIG. 9 depicts an image of a barista milk product formulation.

FIG. 10 depicts an image of a custard product formulation.

FIG. 11 depicts an image of a milk product formulation.

FIG. 12 depicts a graphical plot showing gelation rheology results for a whole animal egg, a RuBisCO baking egg, and a whole RuBisCO egg.

FIG. 13 depicts a graphical plot showing a terminal elastic modulus comparison between a whole animal egg, a RuBisCO baking egg, and a whole RuBisCO egg.

### DETAILED DESCRIPTION

Unless defined otherwise, all technical and scientific terms used herein have the same meaning as commonly understood by those of ordinary skill in the art to which embodiments herein belongs. Any methods, devices and materials similar or equivalent to those described herein can be used in the practice of embodiments herein. The following definitions are provided to facilitate understanding of certain terms used frequently herein and are not meant to limit the scope of the present disclosure. All documents (e. g. patent applications or patents) referred to herein are incorporated by reference in their entirety.

### Definitions

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein can be for the purpose of describing particular embodiments only, and is not intended to be limiting.

Herein, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Unless specifically stated or obvious from context, as used herein, the term “about” in reference to a number or range of numbers is understood to mean the stated number and numbers+/−20% thereof, or 20% below the lower listed limit and 20% above the higher listed limit for the values listed for a range.

Herein, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

Reference herein to “some embodiments,” “an embodiment,” “one embodiment” or “other embodiments” means that a particular feature, structure, or characteristic described in connection with the embodiments can be included in at least some embodiments, but not necessarily all embodiments, of the present disclosure.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited elements or method steps. It is contemplated that any embodiment discussed in this specification can be implemented with respect to any method or composition of the present disclosure, and vice versa. Furthermore, compositions of the present disclosure can be used to achieve methods of the present disclosure.

As used herein, “emulsion activity” is defined as the maximum amount of oil that can be emulsified by a fixed amount of the protein.

The ability of proteins to form gels and stable foams is important in the production of a variety of foods. As used herein, foams refer to structures formed by trapping pockets of gas in a liquid or solid. Proteins in foams contribute to the foam's ability to form small air cells and stability in holding the structure. Foams with a uniform distribution of small air bubbles impart body, smoothness and lightness to the food. The ability of a protein preparation to form a foam is related to its purity, and a purity of at least about 80% may be needed to form a stable foam.

As used herein, gels are soft solids comprising a high amount of an aqueous phase. Protein gels may comprise a three-dimensional network of protein fibers with a continuous liquid phase throughout the matrix. Proteins with higher gelling capacity require less protein to form a gel. The processes disclosed herein may be used to prepare protein preparations with advantageously high purity, foaming capacity, foam stability, and gelling capacity that is suitable for use in food products.

As used herein, “identity,” refers to a relationship between two or more amino acid sequences, as determined by comparing the sequences. In the art, “identity” also refers to the degree of sequence relatedness between amino acid sequences as determined by the match between strings of such sequences. “Identity” can be readily calculated by known methods, including, but not limited to, those described in (Computational Molecular Biology, Lesk, A M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J. Applied Math. 1988, 48: 1073. Preferred methods to determine identity are designed to give the largest match between the sequences tested. Methods to determine identity are codified in publicly available computer programs. The percent identity between two sequences can be determined by using analysis software (e.g., Sequence Analysis Software Package of the Genetics Computer Group, Madison Wis.) that incorporates the Needelman and Wunsch, (J. Mol. Biol., 1970, 48: 443-453,) algorithm (e.g., NBLAST, and XBLAST). The default parameters are used to determine the identity for the polypeptides of the present disclosure, unless stated otherwise.

As used herein, the term “protein” refers to a molecule comprised of amino acid residues, at least two of which are covalently linked by peptide bonds. A protein contains at least two amino acids or amino acid variants, and no limitation is placed on the maximum number of amino acids that can comprise a protein sequence. The term “protein isolate” refers to aa preparation of proteins, wherein the proteins has been substantially separated from non-protein components of a mixture. The “purity” of a protein isolate refers to the amount of protein relative to the total amount of protein preparation. In some embodiments, the purity of the protein isolate is expressed as a percentage of the total dry mass.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of about 0.1% to about 5%″ should be interpreted to include not only the explicitly recited values of about 0.1% to about 5%, but also, unless otherwise stated, include individual values (e.g., about 1%, about 2%, about 3%, about 4%, etc.) and the sub-ranges (e.g., about 0.5% to about 1.1%; about 0.5% to about 2.4%; about 0.5% to about 3.2%, about 0.5% to about 4.4%, and other possible sub-ranges, etc.) within the indicated range. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about, it will be understood that the particular value forms a further disclosure. For example, if the value “about 10” is disclosed, then “10” is also disclosed.

### Overview

Disclosed herein are protein isolate compositions and processes for making protein isolate compositions from plant material. In certain embodiments, provided herein are ribulose-1,5-bisphosphate carboxylase/oxygenase (RuBisCO) protein isolate compositions and processes for making such RuBisCO compositions.

The disclosed compositions can be used in the production of cosmetics, cosmeceuticals, pharmaceuticals, nutraceuticals, supplements, food products, food, beverages, and the like. In some cases, the disclosed compositions can emulate, mimic, or simulate one or more qualities of natural eggs and/or dairy. Natural egg and/or dairy qualities can include binding, moisturizing, leavening, emulsifying, gelling, pH, viscosity, thickening, solubility, and textural characteristics. Other qualities include other benefits that can be derived from consuming or otherwise using natural egg and/or dairy, such as protein content, lipid content, enzymatic activity, nutrients (e.g., essential amino acids), and the like. Accordingly, the compositions disclosed herein can be used to supplement, replace some of, or substitute for the use of natural eggs and/or dairy in cosmetics, cosmeceuticals, pharmaceuticals, nutraceuticals, supplements, food products, food, beverages, amongst other egg and/or dairy containing products.

Such compositions may include isolated and/or purified plant-based proteins, such as ribulose-1,5-bisphosphate carboxylase/oxygenase (RuBisCO) protein isolate. An embodiment of the subject matter described herein includes a plant-based food product that includes a protein isolate, a food component, and a food additive. The protein isolate can comprise a ribulose-1,5-bisphosphate carboxylase/oxygenase (RuBisCO) protein isolate. The protein isolate can comprise a protein content greater than approximately 80%. Furthermore, the protein isolate is free of chlorophyll, is flavorless, and is colorless.

The food additive may be a plasticizer, an oil, a sugar, a flavoring component, a coloring component, a fiber, a soluble salt, a starch, an acid, and/or a wax, among others not explicitly listed herein. The plasticizer may be water, an aqueous polysaccharide solution, an alcohol, a polyalcohol, a glycerol (glycerine), a gum Arabic, a xanthan gum, a guar gum, a locust bean gum, and/or an aqueous solution of carbohydrates, among others not explicitly listed herein. Further, the coloring component may be a turmeric component, among others not explicitly listed herein.

Additionally, the fiber may be pectin, citrus fiber, and/or cellulose, among others not explicitly listed herein. In some examples, the soluble salt may be calcium lactate gluconate, among others not explicitly listed herein. In other examples, the acid may be ascorbic acid and/or citric acid, among others not explicitly listed herein. The oil may be a safflower oil, a coconut oil, a grapeseed oil, and/or a canola oil, among others not explicitly listed herein. Further, the wax may be a naturally-derived wax or a synthetic wax. In examples, the plant-based food product may be a milk replacement product or an egg replacement product.

Methods and compositions for producing plant-based food products are described herein. In some examples, the plant-based food products are non-animal-based replicas of animal-based food products. In other examples, the plant-based food products act as nutraceuticals or carriers for pharmaceutical compositions. In some examples, the plant-based food products may be for human consumption. In other examples, the plant-based food products may be for animal consumption, such as for domesticated or wild animals.

In other examples, the plant-based food products are made to replicate food products, such as to produce an equivalent meat product. The equivalent meat product can be derived from any animal, such as cattle, sheep, pig, chicken, turkey, goose, duck, horse, dog, rabbit, deer, bison, buffalo, boar, snake, pheasant, quail, bear, elk, antelope, pigeon, dove, grouse, fox, wild pig, goat, kangaroo, emu, alligator, crocodile, turtle, groundhog, marmot, possum, partridge, squirrel, raccoon, whale, seal, ostrich, capybara, nutria, guinea pig, rat, mice, vole, any variety of insect or other arthropod, or seafood. Examples of plant-based food products created may include any plant-based food product, including, but not limited to drinks, meats, cheeses, eggs, pastes, pate, etc. The plant-based meat product may be a meat replica and may be made to mimic the look, texture, and taste of the animal-based product, such that is similar to, or indistinguishable from, the given food product.

In some embodiments, isolated and purified protein describe herein are enriched relative to starting material (e.g., plants or other non-animal sources). In some embodiments, the term “isolated and purified” can indicate that the preparation of the protein is at least 60% pure, e.g., greater than 65%, 70%, 75%, 80%, 85%, 90%, 95%, or 99% pure. The isolated and purified protein or the protein isolate may be derived from a non-animal source, such as plants, algae, fungi (e.g., yeast or filamentous fungi), bacteria, or Archaea.

### Protein Isolate Preparations

Processes disclosed herein separates proteins from other compounds found in plant material. Such processes can be considered as purifying or isolating proteins described herein to obtain protein isolates as described herein. For example, the process may remove chlorophyll, volatilized chemical compounds, acids, bases, sugars, salts, and/or lipids. In some embodiments, the processes disclosed herein reduce the amount of chlorophyll, volatilized chemical compounds, acids, bases, sugars, salts, and/or lipids by 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% relative to the source plant material.

In some embodiments, the processes disclosed herein remove chlorophyll from plant material, producing protein isolates that are dechlorophyllized. For instance, in some embodiments, the weight ratio of chlorophyll to protein in the protein isolate is less than about 1:1000, 1:1500, 1:2000, or 1:2500. In some embodiments, the processes disclosed herein reduce the amount of chlorophyll by 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% relative to the source plant material.

In some embodiments, the compositions and processes disclosed herein have decreased or decrease or remove one or more agent(s) that imparts or is associated with one or more organoleptic properties in the purified protein isolates. Non limiting examples of such organoleptic properties include odor (e.g., off-odor or undesirable odor) and taste (e.g., off-taste or undesirable taste). In some embodiments, the compositions and processes disclosed herein have decreased or decrease the one or more agent(s) by 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% relative to the source plant material. In some embodiments, processes decreased herein can produce odorless, tasteless, or both, compositions. In some embodiments, compositions decreased herein can be odorless, tasteless, or both.

Proteins can be extracted from plant material through any known processes. For example, plant material containing protein, such as RuBisCO, can be homogenized and the protein extracted from the pulp and/or liquid. The extract can be further clarified, filtered, and washed to arrive at the described protein isolate. Other extraction processes can include solvent extraction (e.g., using polar solvents, organic solvents, or supercritical fluids), chromatography (e.g., preparative liquid chromatography), clarification, distillation, filtration (e.g., ultrafiltration), recrystallization, and/or solvent-solvent partitioning.

In one aspect of the disclosure, described herein is a process for making a purified protein isolate from a plant material, comprising the steps of: a) providing the plant material in a solution comprising a reducing agent; b) lysing the plant material; c) separating the lysed plant material into a solid phase and a liquid phase, wherein the liquid phase contains soluble protein and chlorophyll; d) coagulating the chlorophyll in the liquid phase by heating it to a first set temperature in no more than about 30 min, then cooling it to a second set temperature in no more than about 30 min, wherein the cooling is initiated when the liquid phase reaches the first set temperature; e) contacting the liquid phase of d) with a flocculant and/or an adsorbent, and mixing for a period of time sufficient to flocculate and/or adsorb chlorophyll in the liquid phase to the adsorbent, thereby forming a flocculated mixture; f) separating the flocculated mixture of e) into a solid phase and a liquid phase; and g) filtering the liquid phase of f) to yield a filtrate containing a purified protein. In some embodiments, the plant material is harvested and cleaned before the process is started. For instance, in some embodiments, the plant material is chemically washed or washed with water prior to processing. In some embodiments, the plant material is washed more than one time prior to processing.

In some embodiments, the plant material is mixed in a solution comprising a reducing agent. Examples of reducing agents suitable for use in the disclosed processes include, but are not limited to, 2-mercaptoethanol (BME), 2-mercaptoethylamine-HCL, sodium sulfite, magnesium sulfite, sodium metabisulfite, sodium bisulfite, cysteine hydrochloride, dithiothreitol (DTT), glutathione, cysteine, tris(2-carboxyethyl)phosphine (TCEP), ferrous ion, nascent hydrogen, sodium amalgam, oxalic acid, formic acid, magnesium, manganese, phosphorous acid, potassium, sodium, and any combination thereof. Said solution may comprise other components to provide beneficial properties to the solution or to the process. Examples such components include buffering agents, chelating agents, protease inhibitors, pH adjustors, and the like.

Lysing can be through any suitable method to disrupt plant material and release cellular contents, such as a plant cell's cytoplasm. Types of lysing described herein include mechanical, chemical, and/or enzymatic lysis. Mechanical lysing encompassed by the processes described herein includes, but is not limited to, mechanical agitation, pressure, grinding, squeezing, shearing, using a blender, using a mill, using a press, a sonicator, a nitrogen burst, ultrasonic energy, by freezing, using a homogenizer, a pulse electric field, a disintegrator, more than one of the foregoing, or any combination thereof. Chemically lysing encompassed by the processes described herein includes, but is not limited to, lysed chemically using one or more of detergents (e.g., ionic, cationic, anionic, sodium dodecyl sulfates, non-ionic, zwitterionic, hypotonic, hypertonic, and isotonic detergents and the like). Chemically lysing encompassed by the processes described herein includes, but is not limited to, using one or more enzymes, such as cellulase and/or pectinase.

Separation of the lysed plant material and/or flocculated mixture into solid phase and a liquid phase may be performed by any suitable solid-liquid separation technique. Suitable solid-liquid separation techniques include but are not limited to: gravity settling, sieving (e.g., circular vibratory separator or a linear/inclined motion shaker), filtration (e.g., dead-end filtration system, using ultrafiltration, using a tangential flow filtration system, or using a plate filter), centrifugation (e.g., disk stack centrifuge, a decanter centrifuge, a continuous centrifuge, or a basket centrifuge), a press (e.g., screw press, a French press, a belt press, a filter press, a fan press, a finisher press, or a rotary press), or decanting (e.g., using a decantor), or any combination thereof.

The process for making the protein isolates described herein can also comprise a step of coagulating components that are undesired (e.g., components that are not protein, such as RuBisCO) using any suitable method to effect coagulation. Examples include, but are not limited to: heat treatment, cooling; addition of one or more salts (e.g., a calcium salt, a magnesium salt, a beryllium salt, a zinc salt, a cadmium salt, a copper salt, an iron salt, a cobalt salt, a tin salt, a strontium salt, a barium salt, a radium salt, calcium chloride, calcium nitrate, or iron carbonate potassium phosphate, calcium chloride, or any combination thereof); addition of quaternary ammonia specie; addition of a polymer based coagulate; electrocoagulation; and the like.

The process for making the protein preparation may also comprise a step of contacting the liquid phase with a flocculant and/or an adsorbent and mixing for a period of time sufficient to flocculate and/or adsorb chlorophyll in the liquid phase to the adsorbent, thereby forming a flocculated mixture. Any suitable process of flocculation can be used and exemplary flocculants may include, but are not limited to, an alkylamine epichlorohydrin, polydimethyldiallylammonium chloride, a polysaccharide (e.g., chitosan), a polyamine, starch, aluminum sulphate, alum, polyacrylamide, polyacromide, or polyethyleneimine. Any suitable adsorbent can be used and exemplary adsorbents may include activated carbon, graphite, silica gel, zeolites, clay, polyethylene, and resins (e.g., ion-exchange resins, size exclusion chromatography (SEC) resins, affinity based resins, or hydrophobicity based resin).

After the separation of the flocculated mixture into a solid phase and a liquid phase, the liquid phase may be filtered to yield a filtrate containing the purified protein. Any suitable method of filtration can be used and include, for example, the use of surface filters, depth filters, by membrane filtration, column filtration, diafiltration, ultrafiltration, tangential flow filtration, filtration with diatomaceous earth, filtration with silt, filtration with activated carbon, and the like.

Liquid phases and/or filtrates can be further sterilized, concentrated, dialyzed, dried, and/or otherwise processed to provide protein isolates for use herein. In some embodiments, liquid phases and/or filtrates may be dried. In some embodiments, drying may be accomplished using a spray dryer, a freeze dryer, drum drying, film drying, bed drying, a flash dryer, or a rotary dryer.

In some embodiments, the purity of protein isolates described herein is at least about 30%, 40%, 50%, 60%, 70%, 80%, 90%, 95%, or more. In some embodiments, the purity of protein isolate described herein is 80% or more. In certain embodiments, protein isolates described herein may contain no more than 50%, 40%, 30%, 20% 10% or less impurities. In certain embodiments, protein isolates described herein may contain no more than 20% 10% or less impurities. In some embodiments, processes described herein produce one or more by-products, such as sodium hydroxide.

Ribulose-1,5-bisphosphate oxygenase (RuBisCO)

Disclosed herein are ribulose-1,5-bisphosphate carboxylase/oxygenase (RuBisCO) protein isolate compositions and processes for making such RuBisCO compositions. In some embodiments, the disclosed compositions and methods relate to the use of RuBisCO protein isolates for plant-based food products, such as plant-based egg replacement products and plant-based milk replacement products.

RuBisCO is considered the most abundant plant protein known and is an enzyme involved in the first major step of carbon fixation, a process by which the atmospheric carbon dioxide is converted by plants and other photosynthetic organisms to energy-rich molecules such as glucose. More specifically, RuBisCO catalyzes the carboxylation of ribulose-1,5-bisphosphate (or “RuBP”). When subjected to heating and other processing, particularly in an aqueous slurry, RuBisCO is known to exhibit various functional properties that may be desirable in a protein source for fiber formation, such as: solubility, viscosity building, gel formation or gelation, water retention, foaming, and emulsifying attributes.

As described herein, RuBisCO is essential in the initial step of the photosynthetic fixation of carbon dioxide and functions to catalyze the carboxylation and/or oxygenation of ribulose-1,5-bisphosphate. Accordingly, the RuBisCO protein isolate can be obtained, derived, purified or isolated from any plant that undergoes photosynthesis.

Compositions disclosed herein can comprise RuBisCO proteins, which can be extracted or isolated from a photosynthesizing plant (e.g., one or more plants of the Nicotiana species) or a photosynthesizing organism (e.g., photosynthetic bacteria). Accordingly, RuBisCO protein isolate used in compositions disclosed herein is obtained from any chlorophyll-containing plant material. In some embodiments, RuBisCO protein isolate used in the disclosed compositions is isolated from a member of the Amaranthaceae, Araceae, Poaceae, Solanaceae, or Apiaceae family. In some embodiments, RuBisCO protein isolate used in the disclosed compositions is isolated from a member of the Lemna, Spirodela, Wolffia, Wolffienlla, Spinacia, Beta, Leymus, Nicotiana, Zea, Solanum, Daucus, Atriplex, Nannochloropsis, Chlorella, Dunaliella, Scenedesmus, Selenastrum, Oscillatoria, Phormidium, Spirulina, Amphora, or Ochromona genus. In some embodiments, RuBisCO protein isolate used in the disclosed compositions can be isolated from one or more of the following species: Lemna aequinoctialis, Lemna disperma, Lemna ecuadoriensis, Lemna gibba (swollen duckweed), Lemna japonica, Lemna minor, Lemna minuta, Lemna obscura, Lemna paucicostata, Lemna perpusilla, Lemna tenera, Lemna trisulca, Lemna turionifera, Lemna valdiviana, Lemna yungensis, Medicago sativa, Nicotiana sylvestris, Nicotiana tabacum, Spinacia oleracea, Beta vulgaris, Leymus arenarius, Zea mays, Daucus carota, Solanum tuberosum, Atriplex lentiformis, Scendesmus dimorphus, Pereskia aculeata, Achnanthes orientalis, Agmenellum spp., Amphiprora hyaline, Amphora coffeiformis, Amphora coffeiformis var. linea, Amphora coffeiformis var. punctata, Amphora coffeiformis var. taylori, Amphora coffeiformis var. tenuis, Amphora delicatissima, Amphora delicatissima var. capitata, Amphora sp., Anabaena, Ankistrodesmus, Ankistrodesmus falcatus, Boekelovia hooglandii, Borodinella sp., Botryococcus braunii, Botryococcus sudeticus, Bracteococcus minor, Bracteococcus medionucleatus, Carteria, Chaetoceros gracilis, Chaetoceros muelleri, Chaetoceros muelleri var. subsalsum, Chaetoceros sp., Chlamydomas perigranulata, Chlorella anitrata, Chlorella antarctica, Chlorella aureoviridis, Chlorella Candida, Chlorella capsulate, Chlorella desiccate, Chlorella ellipsoidea, Chlorella emersonii, Chlorella fusca, Chlorella fusca var. vacuolate, Chlorella glucotropha, Chlorella infusionum, Chlorella infusionum var. actophila, Chlorella infusionum var. auxenophila, Chlorella kessleri, Chlorella lobophora, Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella ovalis, Chlorella parva, Chlorella photophila, Chlorella pringsheimii, Chlorella protothecoides, Chlorella protothecoides var. acidicola, Chlorella regularis, Chlorella regularis var. minima, Chlorella regularis var. umbricata, Chlorella reisiglii, Chlorella saccharophila, Chlorella saccharophila var. ellipsoidea, Chlorella saliva, Chlorella simplex, Chlorella sorokiniana, Chlorella sp., Chlorella sphaerica, Chlorella stigmatophora, Chlorella vanniellii, Chlorella vulgaris, Chlorella vulgaris fo. tertia, Chlorella vulgaris var. autotrophica, Chlorella vulgaris var. viridis, Chlorella vulgaris var. vulgaris, Chlorella vulgaris var. vulgaris fo. tertia, Chlorella vulgaris var. vulgaris fo. viridis, Chlorella xanthella, Chlorella zofingiensis, Chlorella trebouxioides, Chlorella vulgaris, Chlorococcum infusionum, Chlorococcum sp., Chlorogonium, Chroomonas sp., Chrysosphaera sp., Cricosphaera sp., Crypthecodinium cohnii, Cryptomonas sp., Cyclotella cryptica, Cyclotella meneghiniana, Cyclotella sp., Dunaliella sp., Dunaliella bardawil, Dunaliella bioculata, Dunaliella granulate, Dunaliella maritime, Dunaliella minuta, Dunaliella parva, Dunaliella peircei, Dunaliella primolecta, Dunaliella salina, Dunaliella terricola, Dunaliella tertiolecta, Dunaliella viridis, Dunaliella tertiolecta, Eremosphaera viridis, Eremosphaera sp., Ellipsoidon sp., Euglena spp., Franceia sp., Fragilaria crotonensis, Fragilaria sp., Gleocapsa sp., Gloeothamnion sp., Haematococcus pluvialis, Hymenomonas sp., lsochrysis all galbana, lsochrysis galbana, Lepocinclis, Micractinium, Micractinium, Monoraphidium minutum, Monoraphidium sp., Nannochloris sp., Nannochloropsis salina, Nannochloropsis sp., Navicula acceptata, Navicula biskanterae, Navicula pseudotenelloides, Navicula pelliculosa, Navicula saprophila, Navicula sp., Nephrochloris sp., Nephroselmis sp., Nitschia communis, Nitzschia alexandrine, Nitzschia closterium, Nitzschia communis, Nitzschia dissipata, Nitzschia frustulum, Nitzschia hantzschiana, Nitzschia inconspicua, Nitzschia intermedia, Nitzschia microcephala, Nitzschia pusilla, Nitzschia pusilla elliptica, Nitzschia pusilla monoensis, Nitzschia quadrangular, Nitzschia sp., Ochromonas sp., Oocystis parva, Oocystis pusilla, Oocystis sp., Oscillatoria limnetica, Oscillatoria sp., Oscillatoria subbrevis, Parachlorella kessleri, Pascheria acidophila, Pavlova sp., Phaeodactylum tricomutum, Phagus, Phormidium, Platymonas sp., Pleurochrysis camerae, Pleurochrysis dentate, Pleurochrysis sp., Prototheca wickerhamii, Prototheca stagnora, Prototheca portoricensis, Prototheca moriformis, Prototheca zopfii, Pseudochlorella aquatica, Pyramimonas sp., Pyrobotrys, Rhodococcus opacus, Sarcinoid chrysophyte, Scenedesmus armatus, Schizochytrium, Spirogyra, Spirulina platensis, Stichococcus sp., Synechococcus sp., Synechocystisf, Tagetes erecta, Tagetes patula, Tetraedron, Tetraselmis sp., Tetraselmis suecica, Thalassiosira weissflogii, and Viridiella fridericiana. In some embodiments, RuBisCO used in compositions disclosed herein is isolated from one or more sources described herein. In some embodiments, RuBisCO proteins are extracted from an aquatic surface dwelling plant. In some embodiments, RuBisCO proteins are extracted from lemna minor. Lemna minor is a floating freshwater aquatic plant, with one, two, three, or four leaves, each having a single root hanging in the water. Lemna minor has a subcosmopolitan distribution and is native throughout most of Africa, Asia, Europe and North America. It is present wherever freshwater ponds and slow-moving streams occur, except for arctic and subarctic climates.

In some embodiments, a RuBisCO protein isolate can be free from other substances, including naturally occurring substances, such as chlorophyll, and/or substances added to isolate RuBisCO protein from a RuBisCO source, such as a solvent or water. In some embodiments, RuBisCO protein isolate can be chlorophyll-free. In some embodiments, RuBisCO can also be flavorless, tasteless, colorless, and/or uncolored.

Subsequent extraction, RuBisCO proteins may be further processed to improve the purity of the protein sample. In other scenarios, the extracted RuBisCO may undergo further processing (e.g., adjusting the pH, adjusting the heat, etc.) in order to concentrate the extracted proteins.

Additionally, extracted RuBisCO proteins may also be combined with other proteins, where such combination may occur before or after the additional processing described. In some embodiments, RuBisCO protein isolate comprises other proteins, including but not limited to: pea proteins, isolates, and/or concentrates; garbanzo (chickpea) proteins, isolates, and/or concentrates; fava bean proteins, isolates, and/or concentrates; soy proteins, isolates, and/or concentrates; rice proteins, isolates, and/or concentrate; potato proteins, isolates, and/or concentrates; hemp proteins, isolates, and/or concentrates; canola proteins, isolates, and/or concentrates; wheat proteins, isolates, and/or concentrates; corn proteins, isolates, and/or concentrates; zein proteins, isolates, and/or concentrates; rice proteins, isolates, and/or concentrates; oat proteins, isolates, and/or concentrates; potatoes proteins, isolates, and/or concentrates; peanut proteins, isolates, and/or concentrates; legumes/pulses proteins, isolates, and/or concentrates; lentils proteins, isolates, and/or concentrates; or any combinations thereof. RuBisCO protein and other protein combinations. In some examples, RuBisCO and additional proteins may be in a dry form (e.g., powdered, pelletized, or the like). In other examples, the RuBisCO and the additional proteins may be in a liquid form or in a liquid solution.

In some embodiments, the RuBisCO protein isolate comprises protein comprising a sequence that has at least about 60%, 65%, 70%, 75%, 80%, 85%, 90%, 95%, 99%, or about 100% sequence identity with any one of the sequences as set forth in TABLE 1. In some embodiments, the RuBisCO protein isolate comprises protein comprising a sequence that has at least about 60%, 65%, 70%, 75%, 80%, 85%, 90%, 95%, 99%, or about 100% sequence identity with more than one of the sequences as set forth in TABLE 1. In some embodiments, the RuBisCO protein isolate comprises protein comprising a sequence that has at least about 60%, 65%, 70%, 75%, 80%, 85%, 90%, 95%, 99%, or about 100% sequence identity with one or more of the sequences as set forth in SEQ ID NOS: 1 to 10. Provided in TABLE 1 are large and small RuBisCO subunits for various species described herein, including Lemna minor, Nicotiana tabacum, Medicago sativa (alfalfa), Spinacia oleracea (Spinach), and Chlorella vulgaris (green algae).

TABLE 1
RUBISCO SEQUENCES

| SEQ ID | |
|---|---|
| NO: | Sequence |
| 1 | MSPQTETKASAGFKAGVKDYKLNYYTPEYETKDTDILAAFRVTPQPGVPPEEAGA |
| | AVAAESSTGTWTTVWTDGLTSLDRYKGRCYHIEPVAGEENQFIAYIAYPLDLFEE |
| | GSVTNMFTSIVGNVFGFKALRALRLEDLRIPPAYSKTFQGPPHGIQVERDKLNKYG |
| | RPLLGCTIKPKLGLSAKNYGRAVYECLRGGLDFTKDDENVNSQPFMRWRDRFLF |
| | CAEAIYKAQAETGEIKGHYLNATAGTCEEMIKRAVFARELGVPIVMHDYLTGGFT |
| | ANTSLAYYCRDNGLLLHIHRAMHAVIDRQKNHGMHFRVLAKALRMSGGDHVHS |
| | GTVVGKLEGEREMTLGFVDLLRDDFIEKDRSRGIFFTQDWVSMPGVLPVASGGIH |
| | VWHMPALTEIFGDDSVLQFGGGTLGHPWGNAPGAVANRVALEACVKARNEGRD |
| | LAREGNEIIREACNWSPELAAACEVWKEIKFEYEPVDKLDVK |
| 2 | MASSMMASTAAAVARAGPAQSSMVPFNACRSSVPFPATRKANNNLSTLPGNGGR |
| | VSCMQVWPPEGLKKFETLSYLPPLSVEDLAKEVDYLLRNDWVPCIEFSKEGFVYR |
| | ENHASPGYYDGRYWTMWKLPMFGCTDASQVIAEVEEAKKAYPEYFVRIIGFDNK |
| | RQVQCISFIAYKPT |
| 3 | MSPQTETKASVGFKAGVKEYKLTYYTPEYQTKDTDILAAFRVTPQPGVPPEEAGA |
| | AVAAESSTGTWTTVWTDGLTSLDRYKGRCYRIERVVGEKDQYIAYVAYPLDLFE |
| | EGSVTNMFTSIVGNVFGFKALRALRLEDLRIPPAYVKTFQGPPHGIQVERDKLNKY |
| | GRPLLGCTIKPKLGLSAKNYGRAVYECLRGGLDFTKDDENVNSQPFMRWRDRFL |
| | FCAEALYKAQAETGEIKGHYLNATAGTCEEMIKRAVFARELGVPIVMHDYLTGG |
| | FTANTSLAHYCRDNGLLLHIHRAMHAVIDRQKNHGIHFRVLAKALRMSGGDHIH |
| | SGTVVGKLEGERDITLGFVDLLRDDFVEQDRSRGIYFTQDWVSLPGVLPVASGGI |
| | HVWHMPALTEIFGDDSVLQFGGGTLGHPWGNAPGAVANRVALEACVKARNEGR |
| | DLAQEGNEIIREACKWSPELAAACEVWKEIVFNFAAVDVLDK |
| 4 | MAFLIMSSAAAVATGTNAAQASMIAPFTGLKSATSFPVSRKQNLDITSIASNGGRV |
| | QCMQVWPPINKKKYETLSYLPDLSEEQLLREVEYLLKNGWVPCLEFETEHGFVYR |
| | ENNKSPGYYDGRYWTMWKLPMFGCTDATQVLAEVEEAKKAYPQAWIRIIGFDN |
| | VRQVQCISFIAYKPEGY |
| 5 | MSPQTETKATVGFKAGVKDYRLTYYTPDYETKDTDILAAFRVSPQPGVPAEEAG |
| | AAVAAESSTGTWTTVWTDGLTSLDRYKGRCYHIEPVAGEETQFIAYVAYPLDLFE |
| | EGSVNYMFTSIVGNVFGFKALRALRLEDLRIPAAYVKTFQGPPQGIQVERDKLNK |
| | YGRPLLGCTIKPKLGLSAKNYGRAVYECLRGGLDFTKDDENVNSQPFMRWRDRF |
| | LFCAEAIYKAQAETGEIKGHYLNATAGTCEEMMKRAVFARELGVPIVMHDYLTV |
| | GFTANTTLAHYCRDNGLLLHIHRAMHAVIDRQKNHGMHFRVLAKALRMSGGDH |
| | IHAGTVVGKLEGERDITLGFVDLLRDDFIEKDRSRGIFFTQDWVSLPGVLPVASGGI |
| | HVWHMPALTEIFGDDSVLQFGGGTLGHPWGNAPGAVANRVALEACVQARNEGR |
| | DLAREGNEIIREATKWSPELAAACEVWKEIKFEFPAMDN |
| 6 | MALISSAAVTTVNRASSAQANLVAPFTGLKSSAGFPVTKKTNNDITSIASNGGRVN |
| | CMQVWPPVGKKKFETLSYLPPLTEEQLAKEVEYLIRKGWIPCLEFELEKGFVYRE |
| | NHRSPGYYDGRYWTMWRLPLFGATDSSQVLKELADCKAEYPDSFIRIIGFDNVRQ |
| | VQCISFIAHTPKNY |
| 7 | MSPQTETKASVEFKAGVKDYKLTYYTPEYETLDTDILAAFRVSPQPGVPPEEAGA |
| | AVAAESSTGTWTTVWTDGLTNLDRYKGRCYHIEPVAGEENQYICYVAYPLDLFE |
| | EGSVTNMFTSIVGNVFGFKALRALRLEDLRIPVAYVKTFQGPPHGIQVERDKLNK |
| | YGRPLLGCTIKPKLGLSAKNYGRAVYECLRGGLDFTKDDENVNSQPFMRWRDRF |
| | LFCAEALYKAQAETGEIKGHYLNATAGTCEDMMKRAVFARELGVPIVMHDYLT |
| | GGFTANTTLSHYCRDNGLLLHIHRAMHAVIDRQKNHGMHFRVLAKALRLSGGD |
| | HIHSGTVVGKLEGERDITLGFVDLLRDDYTEKDRSRGIYFTQSWVSTPGVLPVASG |
| | GIHVWHMPALTEIFGDDSVLQFGGGTLGHPWGNAPGAVANRVALEACVQARNE |
| | GRDLAREGNTIIREATKWSPELAAACEVWKEIKFEFPAMDTV |
| 8 | MQVWPPLGLKKFETLSYLPPLTTEQLLAEVNYLLVKGWIPPLEFEVKDGFVYREH |
| | DKSPGYYDGRYWTMWKLPMFGGTDPAQVVNEVEEVKKAPPDAFVRFIGFNDKR |
| | EVQCISFIAYKPAGY |
| 9 | MSPQTETKARVGFKAGVKDYRLTYYTPDYQPKDTDILAAFRMTPQPGVPPEEAG |
| | AAVAAESSTGTWTTVWTDGLTSLDRYKGRCYDIEPVPGEENQYIAYIAYPLDLFE |
| | EGSVTNLFTSIVGNVFGFKALRALRLEDLRIPPAYVKTFQGPPHGIQVERDKLNKY |
| | GRGLLGCTIKPKLGLSAKNYGRAVYECLRGGLDFTKDDENVNSQPFMRWRDRFL |
| | FVAEAIYKSQAETGEIKGHYLNATAATAEAMMQRAECAKDLGVPIIMHDYLTGG |
| | FTANTSLSHYCRDNGLLLHIHRAMHAVIDRQRNHGITFRVLAKALRLSGGDHLHS |
| | GTVVGKLEGEREVTLGFVDLMRDDYIEKDRSRGIYFTQDWVSLPGTMPVASGGIH |
| | VWHMPALVEIFGDDACLQFGGGTLGHPWGNAPGAAANRVALEACTQARNEGRD |
| | LAREGGDVIRAACKWSPELAAACEVWKEIKFEFETIDTL |
| 10 | MAALTASLVSCPVAVAAKPARLASPAWPASLSPRRLSPPSPRGPSQTAAAPARCS |
| | CGSPLTTSSSRPSPTCPSDRRPDRQAGRLHHPSGWTPALEFSNAESAYVKDVANIR |
| | FTGGSASCNYYDNRYWAMYKLPMFGCTDASQVLAEIANAVKTFPDSYVRMAAF |
| | DAVRQVQTVAILVHRPASATDYRLPENPQPLIGCTTQLERPQYCKSLANL |

In some embodiments, a RuBisCO protein isolate comprises protein comprising sequence that has at least 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% sequence identity to SEQ ID NO: 1 or 2 or both.

In some embodiments, a RuBisCO protein isolate comprises protein comprising sequence that has at least 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% sequence identity to SEQ ID NO: 3 or 4 or both.

In some embodiments, a RuBisCO protein isolate comprises protein comprising sequence that has at least 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% sequence identity to SEQ ID NO: 5 or 6 or both.

In some embodiments, a RuBisCO protein isolate comprises protein comprising sequence that has at least 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% sequence identity to SEQ ID NO: 7 or 8 or both.

In some embodiments, a RuBisCO protein isolate comprises protein comprising sequence that has at least 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% sequence identity to SEQ ID NO: 9 or 10 or both.

RuBisCO can be composed of 8 large subunits with a molecular mass of about 53 kDalton and 8 small subunits with a molecular mass of about 12 kDalton. Accordingly, in some embodiments, a disclosed RuBisCO protein isolate comprises one or more large subunits, one or more small subunits, or any combination thereof. For example, a RuBisCO protein isolate can include a large subunit, a small subunit or both. In some embodiments, a RuBisCO subunit can comprise an amino acid sequence in any one of the Uniprot records set forth in TABLE 2.

TABLE 2
RUBISCO SUBUNITS

| Subunit | Uniprot Accession Number |
|---|---|
| Large | A9L9A4, A0A3GlN0S3, K4Fl51, A0A3GlMVZ6, H9ALP2, |
| | D6NJG9, Q8WHI2, G8D4W8, D3W4G8, H9ALP3, G0WYT7, |
| | B5WX54, A0A1S7JIB4, A0A482K3Y6, R9S086, |
| | A0A411PP11, D6NJG2, H9AHZ6, Q8WHI1, R4I7N5, |
| | 085040, Q31NB3, P00880, P00875, P00879, O93627, |
| | O03042, P0C2C2, P04718, P00877, P0C512, P04717, |
| | P00876, Pll383, P22849, Q8DIS5, P22859, 085040, |
| | B0Z5Bl, Q31HD8, P22850, |
| Small | P19309, P19308, P19310, P19311, P19312, P00872, P10795, |
| | B3H5S2, P10796, P10797, A0A0S4IJL0, P45686, P04716, |
| | P69249, Q0INY7, Q31NB2, P05348, P08475, P00873, |

In some embodiments, a RuBisCO protein isolate comprises protein comprising sequence that has at least 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% sequence identity to an amino acid sequence as set forth in any one of the records described in TABLE B.

In some embodiments, a disclosed RuBisCO protein isolate comprises protein comprising about 4 to about 478 contiguous amino acids, but is less than the full-length, native or naturally occurring, wild-type ribulose-1,5-bisphosphate carboxylase/oxygenase polypeptide. In some embodiments, a disclosed RuBisCO protein isolate comprises protein comprising about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, about 125, about 126, about 127, about 128, about 129, about 130, about 131, about 132, about 133, about 134, about 135, about 136, about 137, about 138, about 139, about 140, about 141, about 142, about 143, about 144, about 145, about 146, about 147, about 148, about 149, about 150, about 151, about 152, about 153, about 154, about 155, about 156, about 157, about 158, about 159, about 160, about 161, about 162, about 163, about 164, about 165, about 166, about 167, about 168, about 169, about 170, about 171, about 172, about 173, about 174, about 175, about 176, about 177, about 178, about 179, about 180, about 181, about 182, about 183, about 184, about 185, about 186, about 187, about 188, about 189, about 190, about 191, about 192, about 193, about 194, about 195, about 196, about 197, about 198, about 199, about 200, about 201, about 202, about 203, about 204, about 205, about 206, about 207, about 208, about 209, about 210, about 211, about 212, about 213, about 214, about 215, about 216, about 217, about 218, about 219, about 220, about 221, about 222, about 223, about 224, about 225, about 226, about 227, about 228, about 229, about 230, about 231, about 232, about 233, about 234, about 235, about 236, about 237, about 238, about 239, about 240, about 241, about 242, about 243, about 244, about 245, about 246, about 247, about 248, about 249, about 250, about 251, about 252, about 253, about 254, about 255, about 256, about 257, about 258, about 259, about 260, about 261, about 262, about 263, about 264, about 265, about 266, about 267, about 268, about 269, about 270, about 271, about 272, about 273, about 274, about 275, about 276, about 277, about 278, about 279, about 280, about 281, about 282, about 283, about 284, about 285, about 286, about 287, about 288, about 289, about 290, about 291, about 292, about 293, about 294, about 295, about 296, about 297, about 298, about 299, about 300, about 301, about 302, about 303, about 304, about 305, about 306, about 307, about 308, about 309, about 310, about 311, about 312, about 313, about 314, about 315, about 316, about 317, about 318, about 319, about 320, about 321, about 322, about 323, about 324, about 325, about 326, about 327, about 328, about 329, about 330, about 331, about 332, about 333, about 334, about 335, about 336, about 337, about 338, about 339, about 340, about 341, about 342, about 343, about 344, about 345, about 346, about 347, about 348, about 349, about 350, about 351, about 352, about 353, about 354, about 355, about 356, about 357, about 358, about 359, about 360, about 361, about 362, about 363, about 364, about 365, about 366, about 367, about 368, about 369, about 370, about 371, about 372, about 373, about 374, about 375, about 376, about 377, about 378, about 379, about 380, about 381, about 382, about 383, about 384, about 385, about 386, about 387, about 388, about 389, about 390, about 391, about 392, about 393, about 394, about 395, about 396, about 397, about 398, about 399, about 400, about 401, about 402, about 403, about 404, about 405, about 406, about 407, about 408, about 409, about 410, about 411, about 412, about 413, about 414, about 415, about 416, about 417, about 418, about 419, about 420, about 421, about 422, about 423, about 424, about 425, about 426, about 427, about 428, about 429, about 430, about 431, about 432, about 433, about 434, about 435, about 436, about 437, about 438, about 439, about 440, about 441, about 442, about 443, about 444, about 445, about 446, about 447, about 448, about 449, about 450, about 451, about 452, about 453, about 454, about 455, about 456, about 457, about 458, about 459, about 460, about 461, about 462, about 463, about 464, about 465, about 466, about 467, about 468, about 469, about 470, about 471, about 472, about 473, about 474, about 475, about 476, about 477, or about 478 contiguous amino acids of SEQ ID NO: 1, 2, both, one or more large subunits set forth in TABLE B, one or more small subunits set forth in TABLE B, or any combination thereof, but is less than the full-length, native or naturally occurring, wild-type ribulose-1,5-bisphosphate carboxylase/oxygenase polypeptide.

In some embodiments, compositions disclosed herein can comprise about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% protein isolate by dry weight or total weight. In some embodiments, the compositions can comprise about 5% to about 80%, about 6% to about 50%, about 7% to about 40%, about 8% to about 30%, about 9% to about 20%, or about 10% to about 15% protein isolate by dry weight or total weight. In certain embodiments, total weight can include a preservative solution. In some embodiments, description of a measurement by weight is understood to encompass dry weight or total weight.

In some embodiments, compositions disclosed herein can comprise about 1 g to about 100 g of protein isolate. In some embodiments, compositions disclosed herein can comprise about 1 g, about 2 g, about 3 g, about 4 g, about 5 g, about 6 g, about 7 g, about 8 g, about 9 g, about 10 g, about 20 g, about 30 g, about 40 g, about 50 g, about 60 g, about 70 g, about 80 g, about 90 g, about 100 g, or more of a protein isolate, such as a RuBisCO protein isolate.

Compositions described herein can further comprise a plasticizer, an aqueous solution, an acid, a base, a soluble salt, a leavening agent, an additive, optionally a food additive, or any combination thereof. Additives can be synthetic, plant-based, animal-based, or any combination thereof. Examples of food additives described herein include a flavoring component, coloring component, starch, fiber,

In some embodiments, compositions described herein further comprise one or more of a(n): plasticizer, aqueous solution, acid, base, salt, leavening agent, amino acid, nutrient, flavoring component, coloring component, starch, fiber, lecithin, cross-linking agent, lipid, flour, other additives (e.g., a food additive), or any combination thereof.

### Plasticizers

In some examples, the protein isolates, such as RuBisCO protein isolates, may be combined with a plasticizer. As described herein, plasticizers are low molecular weight, non-volatile compounds used as additives or incorporated into other material in order to increase flexibility and dispensability. The process of plasticizing a protein-based polymer or fiber may be affected by the selected plasticizer's molecular weight, as well as the number and position of various hydroxyl groups. Examples of plasticizers include: water, an aqueous polysaccharide solutions, an alcohol, a polyalcohol, a glycerol (glycerine), a gum Arabic, a xanthan gum, a locust bean gum, and/or an aqueous solution of carbohydrates, among others.

Accordingly, in some embodiments, compositions disclosed herein further comprise a plasticizer. In some embodiments, a plasticizer comprises one or more gums. The term “gum” as used herein can refer to materials that act as gelling agents, and can comprise, for example, polysaccharides and/or glycoproteins. Gums used in compositions herein include: xanthan gum, acacia gum, gellan gum, guar gum, locust bean gum, tragacanth gum, carrageenan gum, or a combination thereof.

An amount of the plasticizer added may vary between about 0.01% to about 10% by weight depending on the amount of the protein. In some embodiments, compositions disclosed herein comprise about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3% or more plasticizer by weight. In some embodiments, compositions disclosed herein comprise about 0.1 g, about 0.2 g, about 0.3 g, about 0.4 g, about 0.5 g, about 0.6 g, about 0.7 g, about 0.8 g, about 0.9 g, about 1 g or more of a plasticizer.

Depending on the plasticizer used, the moisture content of the protein isolate compositions may require adjustment. In some embodiments the moisture content may range from about 15% to about 30%. The protein isolate, and optionally, any additional protein(s), in combination with the plasticizer, may be exposed to heat and thermally plasticized. The plasticizing process may require mechanical mixing, which may include any means known in the art.

### Aqueous Solution

Compositions disclosed herein can further comprise an aqueous solution. Compositions herein can comprise about 1 wt % to about 100 wt % by weight of an aqueous solution. In some embodiments, an aqueous solution can comprise water, alcohol, acids, such as citric acid and/or ascorbic acid, or another liquid substance. In some embodiments, the liquid comprise glycerol (glycerine). In certain embodiments, an aqueous solution can comprise a preservative, and may be referred to herein as a preservative solution. Such a preservative solution can comprise water, and one or more acids. Preservative aqueous solutions can be about

Compositions herein can comprise about 1%, about 10%, about 20%, about 30%, about 40%, about 50%, about 60%, about 70%, about 80%, about 90%, about 95%, about 96%, about 97%, about 98%, about 99%, or about 100% by weight of aqueous solution. Compositions herein can comprise about 10 g, about 20 g, about 30 g, about 40 g, about 50 g, about 60 g, about 70 g, about 80 g, about 90 g, about 100 g, about 110 g, about 120 g, about 130 g, about 140 g, about 150 g, about 160 g, about 170 g, about 180 g, about 190 g, about 200 g, about 210 g, about 220 g, about 230 g, about 240 g, about 250 g, about 260 g, about 270 g, about 280 g, about 290 g, about 300 g or more of an aqueous solution.

In some embodiments, an aqueous solution can comprise water, an acid, a base, solutes, soluble salts, or combinations thereof. In some embodiments, solutes can include polysaccharides, such as dissolved polysaccharide to make an aqueous polysaccharide solution.

### Acids/Bases/Soluble Salts

In some embodiments, compositions herein can comprise one or more salts, including soluble salts. Examples of soluble salts include, but are not limited to, calcium lactate gluconate. Compositions herein can comprise about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7% or more by weight of salts, including soluble salts, such as calcium lactate gluconate. Compositions herein can comprise about 0.1 g, about 0.2 g, about 0.3 g, about 0.4 g, about 0.5 g, about 0.6 g, about 0.7 g, about 0.8 g, about 0.9 g, about 1 g, about 2 g, about 3 g, about 4 g, about 5 g, about 6 g, about 7 g, about 8 g, about 9 g, about 10 g or more of a salt, including soluble salt.

In some embodiments, compositions herein can comprise one or more acids or salts thereof. Examples of acids include, but are not limited to, citric acid and ascorbic acid. Compositions herein can comprise about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1% or more weight of an acid, such as citric acid and/or ascorbic acid. Compositions herein can comprise about 0.1 g, about 0.2 g, about 0.3 g, about 0.4 g, about 0.5 g, about 0.6 g, about 0.7 g, about 0.8 g, about 0.9 g, about 1 g, or more of an acid, such as citric acid and/or ascorbic acid.

In some embodiments, compositions herein can comprise one or more bases thereof. Examples of bases include, but are not limited to, potassium carbonate, calcium carbonate, or sodium hydroxide. Compositions herein can comprise about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, or about 1% or more by weight of a base. Compositions herein can comprise about 0.1 g, about 0.2 g, about 0.3 g, about 0.4 g, about 0.5 g, about 0.6 g, about 0.7 g, about 0.8 g, about 0.9 g, about 1 g, or more of a base.

The one or more acids and/or bases, and/or salts can be utilized to modify the pH of the composition, such as the aqueous solution of the composition.

In some embodiments, compositions disclosed herein can have a pH that is similar to the pH of natural eggs or dairy. Natural eggs can have a pH range of about 6-8. Dairy can have a pH range of about 4 to about 8.5. Compositions described herein can have a pH of about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7, about 7.8, about 7.9, about 8, about 8.1, about 8.2, about 8.3, about 8.4, or about 8.5. In some embodiments, compositions described herein can have a pH of less than 4.0. In some embodiments, compositions described herein can have a pH of greater than 8.5. In some embodiments, the pH of compositions can be described herein be about 4 to about 8.5, about 5 to about 7.8, or about 6.4 to about 6.9.

### Leavening Agents

In some embodiments, the compositions can comprise a leavening agent. Examples of leavening agents include, but are not limited to, yeast, sodium bicarbonate (baking soda), baking powder, calcium lactate, a dairy-free calcium lactate, or calcium carbonate. Compositions disclosed herein can comprise about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 2%, about 3%, about 4%, about 5% or more by weight of a leavening agent, such as yeast, sodium bicarbonate (baking soda), baking powder, calcium lactate, a dairy-free calcium lactate, or calcium carbonate. Compositions disclosed herein can comprise about 1 g, about 2 g, about 3 g, about 4 g, about 5 g, about 6 g, about 7 g, about 8 g, about 9 g, about 10 g, about 15 g or more of a leavening agent such as yeast, sodium bicarbonate (baking soda) or baking powder.

### Amino Acids

In some embodiments, compositions described herein can further include one or more amino acids. Compositions disclosed herein can include one or more amino acids comprising: alanine, arginine, asparagine, aspartate, cysteine, cystine, histidine, selenocysteine, methionine, isoleucine, leucine, lysine, phenylalanine, threonine, tryptophan, 5-hydroxytryptophan, valine, glutamate, glutamine, glycine, praline, serine, tyrosine. In some embodiments, compositions described herein can comprise one or more amino acids found in a natural egg and/or dairy. Examples of amino acids found in a natural egg include histidine, isoleucine, leucine, lysine, methionine, phenylalanine, threonine, tryptophan and valine. Examples of amino acids found in dairy include leucine, isoleucine, glutamine, phenylalanine, proline and lysine. Compositions described herein can comprise one or more amino acids in an amount that is similar to the amount found in a comparable sample unit (e.g., as determined by the Atwater system) of dairy, an egg or a portion thereof, such as egg white/albumin, or egg yolk.

### Nutrients

Compositions described herein can comprise one or more nutrients. In some embodiments, compositions described herein can comprise one or more nutrients found in natural eggs and/or dairy. Nutrients found in natural eggs include Vitamin D, Vitamin B12, Choline, Iron, Lutein, Zeaxanthin, Riboflavin (Vitamin B2), Pantothenic Acid (Vitamin B5), Vitamin A, Vitamin E, Phosphorus, Folate, Iodine, and Selenium. Nutrients found in dairy include, calcium, phosphorus, vitamin A, vitamin D (fortified), riboflavin, vitamin B12, protein, potassium, zinc, choline, magnesium, and selenium. Compositions described herein can include one or more nutrients comprising: thiamine, ascorbic acid, L-theanine, acetyl glutathione, riboflavin, pantothenic acid, folic acid, cobalin, Vitamin D, Vitamin B12, Choline, Iron, Lutein, Zeaxanthin, Vitamin A, Vitamin E, Phosphorus, Folate, Iodine, Selenium, zinc, potassium, calcium, or magnesium. In some embodiments, compositions described herein can comprise one or more nutrients in an amount found in a comparable unit (e.g., as determined by the Atwater system) of dairy, an egg or a portion thereof, such as egg white/albumin, or egg yolk.

In some embodiments, the compositions can be fortified with nutrients to provide a comparable or improved nutrient profile comparable to a natural egg and/or dairy.

### Flavoring Components

Compositions described herein can comprise one or more flavoring components. In some embodiments, compositions can comprise one or more natural flavoring components or artificial flavoring components, such as salt, spices, such as turmeric, salt, cinnamon, cloves, allspice, ginger, vanilla, vanilla extract, vanilla flavoring, a sugar (e.g., granulated or powdered sugar), tartar, sweeteners, monosodium glutamate, chocolate chips, coco powder, nuts (e.g., pecans) sulfuric flavoring components, such as black salt, or other flavoring components, such as a flavor masker. In some embodiments, a sugar can be glucose, ribose, maltodextrin, xylose, arabinose, fructose, mannose, galactose, maltose, lactose, a stereoisomer thereof, or combinations thereof. In some embodiments, an artificial flavoring component can be a Givaudan Masker. Compositions disclosed herein can comprise about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight of a flavoring component. about 1 g, about 2 g, about 3 g, about 4 g, about 5 g, about 6 g, about 7 g, about 8 g, about 9 g, about 10 g, about 20 g, about 30 g, about 40 g, about 50 g, about 60 g, about 70 g, about 80 g, about 90 g, about 100 g, about 110 g, about 120 g, about 130 g, about 140 g, about 150 g, about 160 g, about 170 g, about 180 g, about 190 g, about 200 g, about 210 g, about 220 g, about 230 g, about 240 g, about 250 g, about 260 g, about 270 g, about 280 g, about 290 g, about 300 g, about 310 g, about 320 g, about 330 g, about 340 g, about 350 g, about 360 g, about 370 g, about 380 g, about 390 g, about 400 g or more of a flavoring component.

### Coloring Components

Compositions described herein can comprise one or more coloring components. In some embodiments, the compositions can comprise one or more coloring agents. In some embodiments, compositions can comprise one or more natural coloring components or artificial coloring components. In some embodiments, coloring components included in compositions described herein comprise: carotenoids such as beta-carotene, turmeric, annatto, mango yellow, palm-based oils, or combinations thereof. In some embodiments, compositions described herein can comprise about 0.01%, about 0.02%, about 0.03%, about 0.04%, about 0.05%, about 0.06%, about 0.07%, about 0.08%, about 0.09%, about 0.1% or more by weight of a coloring component. In some embodiments, compositions described herein can comprise about 0.01 g, about 0.02 g, about 0.03 g, about 0.04 g, about 0.05 g, about 0.06 g, about 0.07 g, about 0.08 g, about 0.09 g, about 0.1 g, or more of a coloring component.

### Starch

Compositions described herein can further comprise one or more starches, such as, for example, arrowroot starch, cornstarch, tapioca starch, mung bean starch, potato starch, sweet potato starch, rice starch, sago starch, wheat starch. The term “starch” can refer to polysaccharide materials, which when produced in plants, can act as energy stores. Starches can be used to impart thickening and stabilizing properties. In some embodiments, compositions described herein can include about 1%, about 2%, about 3%, about 4%, about 5%, about 6%, about 7%, about 8%, about 9%, about 10%, or more by weight of starch, for example, tapioca starch. In some embodiments, compositions described herein can include about 0.5-20%, about 1-15%, or about 2-10% by weight of starch, for example, tapioca starch. Compositions described herein can include about 1 g, about 2 g, about 3 g, about 4 g, about 5 g, about 6 g, about 7 g, about 8 g, about 9 g, about 10 g or more of a starch.

### Fiber

Compositions described herein can further include fiber. In some embodiments, compositions described herein can include bran, such as a wheat bran, oat bran, corn bran, rice bran, or other bran, psyllium fiber, citrus fiber, bamboo fiber, carrot fiber, oat fiber, cellulose, methylcellulose, crystalline cellulose, pectin, or any combination thereof. In some embodiments, fiber used in composition herein, can be micronized into a fine powder. Compositions described herein can comprise about 0.1%, about 0.2%, about 0.3%, about 0.4%, about 0.5%, about 0.6%, about 0.7%, about 0.8%, about 0.9%, about 1%, about 2%, about 3% or more by weight of fiber. Compositions described herein can comprise about 0.1 g, about 0.2 g, about 0.3 g, about 0.4 g, about 0.5 g, about 0.6 g, about 0.7 g, about 0.8 g, about 0.9 g, about 1 g, about 2 g, about 3 g or more of fiber.

### Lecithin

Compositions described herein can further include lecithin. Lecithins can be yellow, brownish, fatty substances that are found in animal and plant tissues, and animal product tissues, such as egg yolk. Lecithin can act as an emulsifier, and can have a similar fat profile to that of natural eggs. Lecithins can also be non-allergenic. In some embodiments, compositions described herein can comprise lecithin, such as plant-based lecithin. Examples of lecithins included in compositions disclosed herein include garbanzo lecithin, fava bean lecithin, soy lecithin, sunflower lecithin, canola lecithin, or a combination thereof. In some embodiments, compositions described herein can comprise about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5% or more by weight of lectin. In some embodiments, compositions described herein can comprise about 0.01 g, about 0.02 g, about 0.03 g, about 0.04 g, about 0.05 g, about 0.06 g, about 0.07 g, about 0.08 g, about 0.09 g, about 0.1 g, about 0.2 g, about 0.3 g, about 0.4 g, about 0.5 g or more of a lectin.

### Crosslinking Agent

In further examples, one or more other additives may also be included in the compositions described herein, such as glossing agents or crosslinking agents. A crosslinking agent may be used to promote desirable changes in a disclosed composition's physical properties, such as causing a polymer to: harden, have an increased melting temperature, etc. Crosslinks may also be formed by chemical reactions under heat, pressure, and/or pH changes. Example crosslinking agents included in compositions described herein include: calcium chloride, calcium phosphate, calcium sulfate, polysaccharides, formaldehyde, glutaraldehyde, dimethyl adipimidate, dimethyl suberimidate, glyoxal, and/or maleic anhydride, a gelling agent, a wax, among others. In some embodiments, wax may also be added to the mixture to provide additional stability to compositions disclosed herein. The wax may include a naturally-derived wax or a synthetic wax.

Compositions described herein can comprise about 0.1%, about 0.2%, about 0.3%, about 0.4%, about 0.5%, about 0.6%, about 0.7%, about 0.8%, about 0.9%, about 1%, about 1%, about 2%, about 3%, about 4%, about 5%, about 6%, about 7%, about 8%, about 9%, about 10%, or more by weight of a cross-linking agent. Compositions described herein can comprise about 0.1 g, about 0.2 g, about 0.3 g, about 0.4 g, about 0.5 g, about 0.6 g, about 0.7 g, about 0.8 g, about 0.9 g, about 1 g, about 1 g, about 2 g, about 3 g, about 4 g, about 5 g, about 6 g, about 7 g, about 8 g, about 9 g, about 10 g, or more of a cross-linking agent.

### Lipids

Compositions disclosed herein can further comprise one or more lipids. In some embodiments, the lipid is a solid lipid, an oil, butter or fat. In some embodiments, compositions disclosed herein can comprise a plant-based lipid, an animal-based lipid, a synthetic lipid, or combinations thereof. In some embodiments, compositions disclosed herein can comprise grapeseed oil, canola oil, sunflower oil, safflower oil, butter, peanut butter, cashew butter, coconut butter, coconut mana, coco butter, soy bean oil, coconut oil, corn oil, olive oil, peanut oil, palm oil, oil from beans, such as garbanzo beans or fava beans, and the like. Compositions disclosed herein can comprise about 1%, about 2%, about 3%, about 4%, about 5%, about 7.5%, about 10%, about 15%, about 20%, about 25% or more weight of a liquid. Weight can be by dry weight or total weight. Compositions disclosed herein can comprise about 1 g, about 2 g, about 3 g, about 4 g, about 5 g, about 6 g, about 7 g, about 8 g, about 9 g, about 10 g, about 20 g, about 30 g, about 40 g, about 50 g, about 60 g, about 70 g, about 80 g, about 90 g, about 100 g, about 110 g, about 120 g, about 130 g, about 140 g, about 150 g, about 160 g, about 170 g, about 180 g, about 190 g, about 200 g, about 210 g, about 220 g, about 230 g, about 240 g, about 250 g, about 260 g, about 270 g, about 280 g, about 290 g, about 300 g, about 310 g, about 320 g, about 330 g, about 340 g, about 350 g, about 360 g, about 370 g, about 380 g, about 390 g, about 400 g or more of a lipid.

### Flour and Dry Mixes

Compositions disclosed herein can comprise one or more flours or mixes. Flour can be powder ground from grains, seeds, roots, or other sources. Flours can have a high starch content which can impart thickening and binding properties, and may provide moisture content. In some embodiments, compositions described herein can further include one or more flours, the flours comprising: all-purpose flour, unbleached flour, bleached flour, bread flour, self-rising flour, wheat flour, cake flour, acorn flour, almond flour, amaranth flour, atta flour, rice flour, buckwheat flour, cassava flour, chestnut flour, chuño flour, coconut flour, corn (maize) flour, hemp flour, maida flour, mesquite flour, nut flour, peanut flour, potato flour, rice flour, rye flour, tapioca flour, teff flour, soy flour, peanut flour, arrowroot flour, taro flour, acorn flour, bean flours such as, e.g., soy flour, garbanzo flour, fava bean flour, pea flour; or other flour. In some embodiments, the one or more flours are selected from Sorghum, White sorghum, Soy bean, Millet, Vallarta, Stueben, Green fagelot, Black beluga, Black calypso, Chana dal, Amaranth, Lentil, Red lentil, Black lentil, Golden lentil, Do pung-style lentil, Sprouted green lentil, Sweet brown rice, Navy bean, Red bean, Pink bean, Canellini bean, Giant white lima bean, Christmas lime bean, Baby lima bean, Mung bean, Peeled fava bean, Good mother stellard bean, Cranberry chorlottis bean, Santa maria pinguinto bean, Brown tepary bean, Black turtle bean, Yellow slit pea, Canadian yellow pea, Black turtle beans, Brown teff flour, Rye flour, Quinoa flour, Potato flour, White rice flour, Brown rice flour, Oat flour, Buckwheat flour, Whole grain corn flour, Stone ground cornmeal, Pre-cooked split pea, Pre-cooked garbanzo flour, Arrowroot powder, and Potato starch. Dry mixes include pre-formulated mixes such as yellow cake mix, brownie mix, cookie mix, and the like.

In some embodiments, the composition can comprise about 10%, about 20%, about 30%, about 40%, about 50%, about 60%, about 70%, about 80%, about 85% about 90%, about 95%, or about 100% by weight of flour. In some embodiments, the composition can comprise about 1-30%, about 10-40%, about 30-70%, about 50-99%, about 60-95%, about 70-90% by weight of flour or dry mix. In some embodiments, the composition can comprise about 10 g to about 500 g flour or dry mix. In some embodiments, the composition can comprise about 10 g, about 20 g, about 30 g, about 40 g, about 50 g, about 60 g, about 70 g, about 80 g, about 90 g, about 100 g, about 110 g, about 120 g, about 130 g, about 140 g, about 150 g, about 160 g, about 170 g, about 180 g, about 190 g, about 200 g, about 210 g, about 220 g, about 230 g, about 240 g, about 250 g, about 260 g, about 270 g, about 280 g, about 290 g, about 300 g, about 310 g, about 320 g, about 330 g, about 340 g, about 350 g, about 360 g, about 370 g, about 380 g, about 390 g, about 400 g, about 410 g, about 420 g, about 430 g, about 440 g, about 450 g, about 460 g, about 470 g, about 480 g, about 490 g, about 500 g, or more of flour or dry mix. In some embodiments, the flour is cold-milled.

### Further Embodiments

Compositions described herein can further comprise one or more ingredients. In some embodiment, compositions described can include one or more ingredients, the one or more ingredients comprising a synthetic ingredients, an ingredients from an animal, or a plant based ingredient.

A synthetic ingredient can include one or more artificial flavorings and ingredients, such as chocolate chips, and the like. Compositions can comprise 20 g to 350 g or more synthetic ingredient. In some embodiments, the composition can comprise about 20 g, about 30 g, about 40 g, about 50 g, about 60 g, about 70 g, about 80 g, about 90 g, about 100 g, about 110 g, about 120 g, about 130 g, about 140 g, about 150 g, about 160 g, about 170 g, about 180 g, about 190 g, about 200 g, about 210 g, about 220 g, about 230 g, about 240 g, about 250 g, about 260 g, about 270 g, about 280 g, about 290 g, about 300 g, about 310 g, about 320 g, about 330 g, about 340 g, about 350 g, or more synthetic ingredient.

A plant-based ingredient can include one or more vegetables (e.g., carrot), fruits (e.g., strawberries), roots (e.g., taro), nut (e.g., pecans) and the like. Compositions can comprise 20 g to 350 g or more plant-based ingredient. In some embodiments, the composition can comprise about 20 g, about 30 g, about 40 g, about 50 g, about 60 g, about 70 g, about 80 g, about 90 g, about 100 g, about 110 g, about 120 g, about 130 g, about 140 g, about 150 g, about 160 g, about 170 g, about 180 g, about 190 g, about 200 g, about 210 g, about 220 g, about 230 g, about 240 g, about 250 g, about 260 g, about 270 g, about 280 g, about 290 g, about 300 g, about 310 g, about 320 g, about 330 g, about 340 g, about 350 g, or more plant-based ingredient.

An animal ingredient can include one or more eggs, dairy (e.g., milk, butter, etc.), meat from an animal and the like. Compositions can comprise 20 g to 350 g or more animal ingredient. In some embodiments, the composition can comprise about 20 g, about 30 g, about 40 g, about 50 g, about 60 g, about 70 g, about 80 g, about 90 g, about 100 g, about 110 g, about 120 g, about 130 g, about 140 g, about 150 g, about 160 g, about 170 g, about 180 g, about 190 g, about 200 g, about 210 g, about 220 g, about 230 g, about 240 g, about 250 g, about 260 g, about 270 g, about 280 g, about 290 g, about 300 g, about 310 g, about 320 g, about 330 g, about 340 g, about 350 g, or more animal ingredient.

In further embodiments, the RuBisCO protein may be a RuBisCO protein isolate that comprises a protein content of greater than about 80%, a protein content of greater than about 85%, a protein content of greater than about 90%, or a protein content of greater than about 95%.

In some embodiments, the plant-based food product further includes a second isolated and purified protein, and/or a seasoning agent, a coloring agent, a flavoring agent, a gelling agent, a sugar, or a fiber.

In some embodiments, the plant-based food product further includes a second isolated and purified protein, and/or a seasoning agent, a coloring agent, a flavoring agent, a gelling agent, a sugar, or a fiber.

### Formulations

Compositions described herein can comprise a protein isolate and a plasticizer, wherein a ratio by weight of the plasticizer to the protein isolate is about 1:5, about 1:10, about 1:15, about 1:20, about 1:25, about 1:30, about 1:35, about 1:40, about 1:45, about 1:50, about 1:55, about 1:60, about 1:65, about 1:70, about 1:75, about 1:80, about 1:85, about 1:90, about 1:95, about 1:100, about 1:105, about 1:110, about 1:115, about 1:120, about 1:125, about 1:130, about 1:135, about 1:140, about 1:145, or about 1:150 (plasticizer:protein isolate). In some embodiments, compositions described herein can comprise a protein isolate and a plasticizer, wherein a ratio by weight of the plasticizer to the protein isolate is about 1:5 to about 1:150, 1:25 to about 1:100, or about 1:40 to about 1:50. In some embodiments, the protein isolate is a RuBisCO protein isolate.

Compositions described herein can comprise a protein isolate and an aqueous solution, wherein a ratio by weight of the protein isolate to the aqueous solution is about 1:4, 1:5, about 1:10, about 1:15, about 1:20, about 1:25, about 1:30, about 1:35, about 1:40, about 1:45, about 1:50, about 1:55, about 1:60, about 1:65, about 1:70, about 1:75, about 1:80, about 1:85, about 1:90, about 1:95, or about 1:100, (protein isolate:aqueous solution). In some embodiments, compositions described herein can comprise a protein isolate and an aqueous solution, wherein a ratio by weight of the plasticizer to the protein isolate is about 1:4 to 1:20, about 1:5 to 1:8, or about 1:8 to 1:20. In some embodiments, the protein isolate is a RuBisCO protein isolate.

Compositions described herein can be liquid or semi-liquid formulations. In certain embodiments, processes described herein can produce liquid or semi-liquid formulations.

Compositions described herein can be dry formulations. In certain embodiments, processes described herein can produce dry formulations. Any suitable drying method can be used to produced compositions described herein, and include but are not limited to: sublimation, such as freeze drying (lyophilization); evaporation such as spray drying and fluidized bed drying; or precipitation such as supercritical fluid technology. In some embodiments, described herein are processes to produce a dried formulation comprising drying a protein isolate solution by lyophilisation. In some embodiments, described herein are processes to produce a dried formulation comprising drying a protein isolate solution by spray-drying. In certain embodiments, a protein isolate solution can be a solution comprising protein isolate. In some embodiments the protein isolate solution comprises a preservative or a preservative solution, and one or more additives described herein, one or more excipient, or any combination thereof. Excipients include surfactants, and other suitable excipient to facilitate the drying process. In some embodiments, the protein isolate solution is obtained from processing plant material to extract protein isolate preparations.

In some embodiments, compositions described herein can be prepared by dry blending. In some embodiments, compositions described herein can be processed using an inline high-shear mixer, cell disruption, liquid chromatography, including HPLC, sonication, and/or rotor-stator mixing technology. In some embodiments, the mixer can have a pump capability of at least about 500 gallons/minute, with a throughput of 50 gallons/minute. In some embodiments, the sonicator can have a throughput of at or above 5 gallons/minute. In other embodiments, compositions described herein can be prepared using standard home kitchen materials, e.g., a kitchen scale, mixing bowl, utensils, blender, or food processor. In some embodiments, compositions described herein can be stored as a dry material. In some embodiments, compositions described herein can be stored as a liquid or semi-liquid material.

Described herein are products, such as food products or plant-based food products, comprising compositions described herein. Products, such as food products, described herein can comprise liquid, semi-liquid, or dry compositions as described herein. In certain embodiments, products comprising compositions described herein include a: cosmetic, cosmeceutical, pharmaceutical, nutraceutical, supplement, food, beverage, egg-replacement products, dairy-replacement products, egg-containing products (e.g., meringues, burgers, meatballs, meatloaf, mayonnaise, dressings, baked goods, dough-based goods, shampoos, facial washes, skin masks, creams, films, encapsulates, vaccines, etc.) dairy-containing products (e.g., ice cream, dressings, sauces, yogurt, custard, sour cream, coffee products, milk, face-creams, lotions, baked goods, dough-based goods, etc.), baked goods (e.g., cake, muffins, cupcakes, brownies, cookies, pies, scones, biscuits, souffles, custards, pastries, tarts, etc.), dough-based goods (e.g., pancakes, waffles, pasta products, breads, crackers, pretzels, etc.), or any combination thereof.

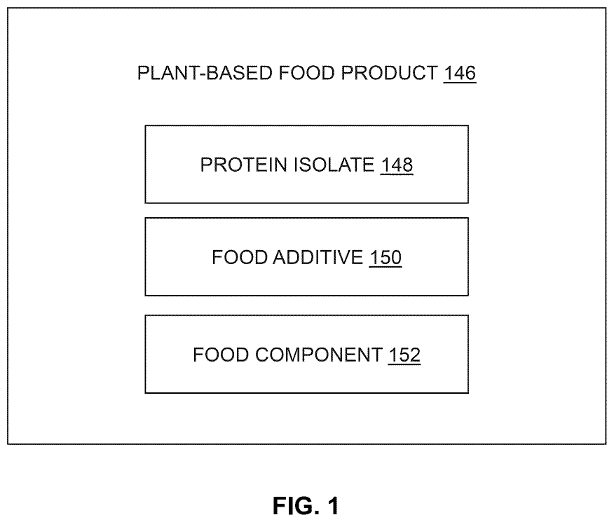

As shown in FIG. 1, a plant-based food product 146 is described and depicted that includes a protein isolate 148, a food additive 150 and optionally a food component 152. In examples, the protein isolate 148 comprises a ribulose-1,5-bisphosphate carboxylase/oxygenase (RuBisCO) protein isolate. Moreover, the RuBisCO protein isolate comprises a protein content greater than approximately 80%. The RuBisCO protein isolate is free of chlorophyll and is flavorless and colorless.

Additionally, the RuBisCO protein isolates described herein emulsify added fats without the addition of other emulsifiers. Other egg replacers on the market either rely on ingredients that are not considered “clean-label” or are low in protein. For example, the gelation behavior one product is dependent on the addition of transglutaminase, while another product utilizes several gelling agents, especially methylcellulose. A flaxseed product on the market, and other starch based egg replacers, do not have the protein content inherent in an animal egg. The essential amino acid content of the RuBisCO protein isolate described herein is on par with animal eggs, meaning that it not only replicates the texture, but also replicates the nutritional profile of the animal egg with no cholesterol. The RuBisCO egg formulations several examples disclosed herein bind water and oil better than other plant-based protein and polysaccharide systems, based on lab studies done on these properties, meaning that use of RuBisCO in baked goods should have higher water retention, better chew and mouthfeel, and better cohesiveness than other egg substitutes.

Compositions described herein can be used as a product, for example cosmetic, cosmeceutical, pharmaceutical, nutraceutical, supplement, food, or beverage product. In some embodiments, compositions described herein are a plant-based food product. Example recipes include, without limitation: Chocolate Chip Cookies (227 grams salted butter softened, 200 grams white (granulated) sugar, 220 grams light brown sugar, 9 grams pure vanilla extract, 100 grams homogenized eggs/RuBisCO Baking Egg, 360 grams all-purpose flour, 6 grams baking soda, 2 grams baking powder, 350 g chocolate chips), Carrot Cake (298 grams vegetable oil, 397 grams granulated white sugar, 6 grams salt, 7 grams cinnamon, 2 grams ground ginger, 1 gram ground cloves, 10 grams baking soda, 200 grams homogenized egg/RuBisCO Baking Egg, 240 grams all-purpose flour, 326 grams grated carrots, 170 grams chopped pecans, toasted), Gingerbread (145 grams unsalted butter, 150 grams light brown sugar, 200 grams molasses, 50 grams homogenized egg/RuBisCO Baking Egg, 9 grams pure vanilla extract, 438 grams all-purpose flour, 6 grams baking soda, 2 grams baking powder, 7 grams ground ginger, 7 grams ground cinnamon, 4 grams ground allspice, 4 grams ground cloves), Royal Icing (480 grams confectioner's sugar, sifted, 16.50 grams RuBisCO protein, 127 grams water), Chocolate Cake (227 grams all purpose flour, 75 grams unsweetened cocoa powder, 10 grams baking powder, 10 grams baking soda, 5 grams salt, 410 grams white granulated sugar, 110 grams homogenized egg/RuBisCO Baking Egg, 250 grams 2% milk or oat milk, 125 grams vegetable oil, 18 grams pure vanilla extract, 250 grams boiling water), Chocolate Cake—Gluten Free (320 grams granulated sugar, 285 grams all purpose gluten-free flour blend, 65 grams unsweetened cocoa powder, 6 grams baking powder, 8 grams baking soda, 6 grams salt, 110 grams homogenized egg/RuBisCO Baking Egg, 260 grams 2% milk or oat milk, 110 grams vegetable oil, 4 grams vanilla extract, 150 grams boiling water), and Choux pastry (84 grams butter, 235 grams water, 2 grams salt, 8 grams sugar, 128 grams all-purpose flower, 200 grams RuBisCO Baking Egg). RuBisCO Baking Egg referred to herein is consistent with as described in the examples.

As shown in FIG. 1, a plant-based food product 146 is described and depicted that includes a protein isolate 148, a food additive 150 and optionally a food component 152. In examples, the protein isolate 148 comprises a ribulose-1,5-bisphosphate carboxylase/oxygenase (RuBisCO) protein isolate. Moreover, the RuBisCO protein isolate comprises a protein content greater than approximately 80%. The RuBisCO protein isolate is free of chlorophyll and is flavorless and colorless.

The food additive 150 includes a plasticizer, an oil, a sugar, a soluble salt, a starch, an acid, a wax, a fiber, a flavoring component, and/or a coloring component, among other components not explicitly listed herein. In examples, the plasticizer is water, an aqueous polysaccharide solutions, an alcohol, a polyalcohol, a glycerol (glycerine), a gum Arabic, a xanthan gum, a guar gum, a locust bean gum, and/or an aqueous solution of carbohydrates, among other plasticizers not explicitly listed herein. The oil is a safflower oil, a grapeseed oil, a coconut oil, and/or a canola oil, among other components not explicitly listed herein.

The soluble salt includes calcium lactate gluconate, among other salts not explicitly listed herein. The acid includes ascorbic acid and/or citric acid, among other acids not explicitly listed herein. The wax may include a naturally-derived wax and/or a synthetic wax. The fiber is a citrus fiber, pectin, and/or cellulose, among other components not explicitly listed herein. The coloring component comprises turmeric. However, it should be appreciated that these examples are provided for illustrative purposes only and other components not explicitly listed are contemplated by Applicant.

Specifically, in examples disclosed herein, the protein isolate 148 comprises a RuBisCO protein isolate, that functions as a functional binding ingredient or component in various food applications. As such, the RuBisCO protein may be used as an emulsifier, a gelling agent, and/or fat binding agent in multiple plant-based food applications. It should be appreciated that the gums and starches are added to the plant-based food product to enhance the texture for different applications, but the gelation behavior is primarily driven by the RuBisCO protein isolate. The turmeric is added to the plant-based food product to give the desired bright yellow color to the product.

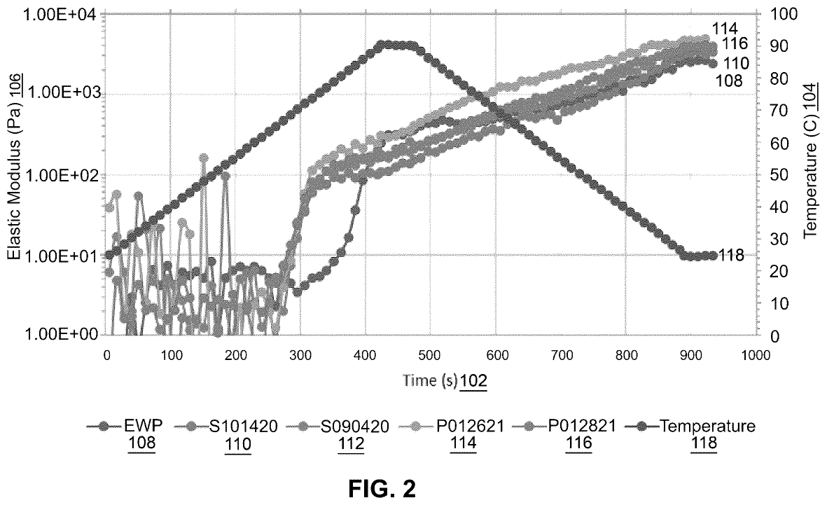

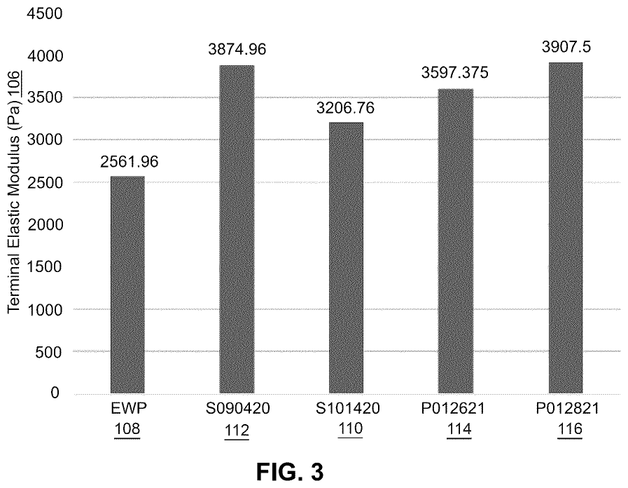

In some embodiments, provided herein are RuBisCO protein isolates for various egg replacement applications. Such examples showcase the native gelling capabilities and emulsifying properties of RuBisCO. Specifically, the RuBisCO protein isolate gels upon heating and forms a gel that has similar or slightly higher strength to that of egg white. These results are depicted in FIG. 2 and FIG. 3.

In some embodiments, compositions described herein can replace a part of, or all of an animal-derived substance in an animal-based product, such as an egg-based product or a dairy-based product.

Egg-based products comprise one or more eggs, components or portions thereof. In some embodiments, compositions described herein can replace a part of or substitute entirely the egg in an egg-based product, such as, baked goods, breakfast foods, and the like. In some embodiments, compositions described herein can replace whole eggs or a part of the egg on a 1:5 basis by weight, wherein 1 weight unit of the compositions replaces 5 weight units of eggs. In other embodiments, the disclosed compositions replaces whole eggs on a 10:1, 9:1, 8:1, 7:1, 6:1, 5:1, 4:1, 3:1, 2:1, or 1:1 basis by weight. In other embodiments the disclosed compositions replace whole eggs or a part of an egg on a 1:10, 1:9, 1:8, 1:7, 1:6, 1:5, 1:4, 1:3, 1:2, or 1:1 basis by weight.

Dairy products, or dairy-based products, as used interchangeably herein, comprise milk or foodstuffs produced from milk. In some embodiments, compositions described herein can replace a part of or substitute entirely the dairy in a dairy-based product, such as, milk, milk cream, sour cream, creme fraiche, buttermilk, cultured buttermilk, milk powder, condensed milk, evaporated milk, butter, cheese, cottage cheese, cream cheese, yogurt, ice cream, frozen custard, frozen yogurt, gelato, via, piima, filmjolk, kajmak, kephir, villi, kumiss, airag, ice milk, casein, ayran, lassi, khoa, or combinations thereof. Dairy-based products can also include products that utilize dairy, and include, but are not limited to, baked goods, sauces, stews, and the like. In some embodiments, compositions described herein can replace all of or part of dairy on a 1:5 basis by weight, wherein 1 weight unit of the compositions replaces 5 weight units of dairy. In other embodiments, the disclosed compositions replaces dairy on a 10:1, 9:1, 8:1, 7:1, 6:1, 5:1, 4:1, 3:1, 2:1, or 1:1 basis by weight. In other embodiments the disclosed compositions replace all of or part of dairy on a 1:10, 1:9, 1:8, 1:7, 1:6, 1:5, 1:4, 1:3, 1:2, or 1:1 basis by weight.

In some embodiments, provided herein are plant-based food products comprising a RuBisCO protein isolate that possesses clean label gelation properties, a high emulsification ability with no added emulsifier, a complete protein nutritional profile as compared to traditional food products (e.g., a nutrition formulation comparable to that of an animal egg), an ability to function as a 1:1 egg replacer in baking, enhanced binding of water and fat in baking formulations, and the same texture in baked goods as compared to an animal egg.

In some embodiments, provided herein are RuBisCO protein isolates to form milk replacement products. The RuBisCO milk has excellent properties while maintaining protein, fat, and carbohydrate levels similar to animal milk. This is a benefit over other plant-based milks (soy, oat, almond, etc.), which usually have much lower protein content and a much higher carbohydrate content. Furthermore, traditional and commercial non-dairy milks are highly processed foods, and almost all plant milks contain food additives, such as: carrageenan, gums (such as xanthan, locust bean, guar and gellan), and/or isolated food starches and fibers to thicken and stabilize them into a more palatable consistency. Additionally, unless a plant-based milk is completely unsweetened, all plant-based milk products contain added sugar. The physiological composition of plant-based dairy alternatives also brings processing challenges, as the composition and structure of raw milk are significantly different from plant-derived materials.

As such, in examples, the plant-based food product 145 is a whole milk product, a barista milk product, a custard, a heavy cream, a whipped cream, a half-and-half product, a yogurt, a sour cream, or a cream cheese, among others not explicitly listed herein. In some examples, the plant-based food product 146 also includes the food component 108, such as a cake mix.

The essential amino acid profile of the milk replacement product described herein is similar to milk and has the same Protein Digestibility Corrected Amino Acid Score (PDCAAS) score, something almost all plant-based milks cannot achieve. A PDCAAS score is a score based on evaluating the quality of a protein based on both the amino acid requirements of humans and their ability to digest it. Moreover, the flavor of the RuBisCO protein is low, allowing for easy masking with low flavor additives. In some embodiments, no color additives are required for the RuBisCO milk products described. The application of RuBisCO protein isolates to these milk products highlights the gelling and excellent emulsification properties of RuBisCO. Stable emulsions that are similar in color to animal milk can be made with the RuBisCO protein isolate with no added emulsifier.

Additionally, heat coagulation can allow for the transformation of milk formulations in gel-like milk formulations. This would allow for the emulation of yogurt, sour cream, etc. while still maintaining a high protein and low carb formulation. In some embodiments, high levels of saturated fats (coconut oil, cocoa butter, etc.) are not added to food product composition described herein to achieve texturing. In some embodiments, a relatively high amount of coconut oil in a sour cream formulation described in the Examples section (˜9.6% saturated fat), while other commercial vegan sour creams range from 5-10% saturated fat. Additionally, the custard formulation in the Examples section has no saturated fat, but a high viscosity and cream texture. In some examples, gum may be used as a viscosity modifier, but it is an emulsifier. In some embodiments, RuBisCO is provided in a composition as the emulsifying agent and not lecithin, or other emulsifiers.

As such, the benefits of the RuBisCO milk replacement products described herein include: a clean label milk formulation, a high emulsification ability with no added emulsifier, a complete protein nutritional profile, a nutritional formulation identical to animal milk, a milk flavor that may be covered up easily, an ability for the milk replacement product to function as a 1:1 milk replacer in baking, and a thermal gelation to make cream.

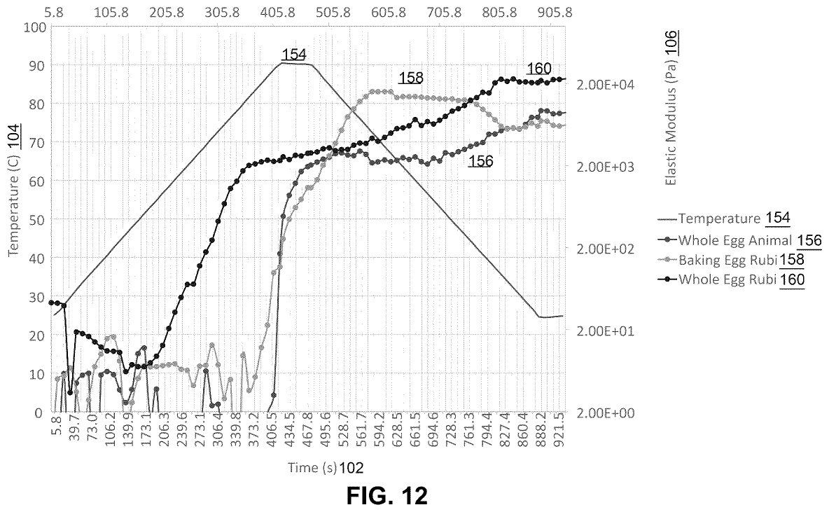

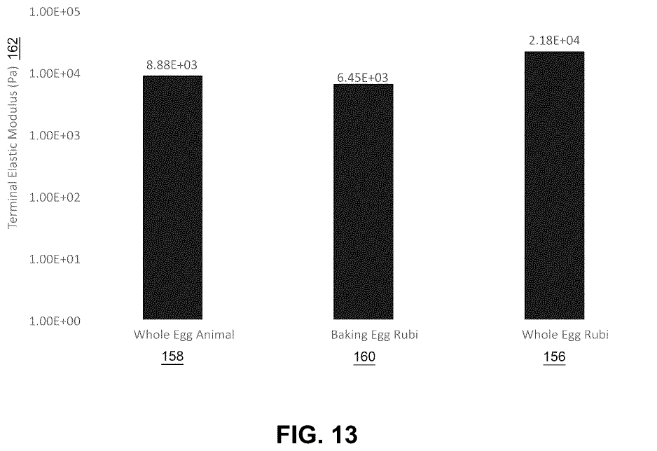

FIG. 12 depicts a graphical plot showing gelation rheology results and FIG. 13 depicts a graphical plot showing a terminal elastic modulus comparison between a whole animal egg, a RuBisCO baking egg, and a whole RuBisCO egg, according to at least some embodiments disclosed herein.

Similar to FIG. 2, FIG. 12 includes an x-axis 102 associated with time in seconds, a y-axis 104 associated with temperature in degrees Celsius, and another y-axis 106 associated with an elastic modulus (in Pa). A temperature 154, as well as data points for a whole animal egg 158, a RuBisCO baking egg 160, and a whole RuBisCO egg 156 are plotted. Moreover, FIG. 13 depicts a y-axis 162 associated with a terminal elastic modulus (in Pa). Values associated with the whole animal egg 158, the RuBisCO baking egg 160, and the whole RuBisCO egg 156 are plotted in FIG. 13.

### Exemplary Embodiments

Provide herein are compositions, wherein the compositions comprise: a ribulose-1,5-bisphosphate carboxylase/oxygenase (RuBisCO) protein isolate; an aqueous solution; and a plasticizer, wherein a ratio by weight of the plasticizer to the RuBisCO protein isolate is about 1:5 to 1:200, or wherein the plasticizer is present in amount of up to about 8% by weight. Further provided here are compositions wherein a ratio by weight of the plasticizer to the RuBisCO protein isolate is about 1:40 to 1:50. Further provided here are compositions wherein the plasticizer is present in amount of up to about 6.5% by weight. Further provided here are compositions wherein n the plasticizer is present in amount of up to about 5% by weight. Further provided here are compositions wherein the plasticizer is present in amount of 0.25% to 1% by weight. Further provided here are compositions wherein the plasticizer comprises a gum. Further provided here are compositions wherein the gum is gum Arabic, a xanthan gum, a guar gum, or a locust bean gum. Further provided here are compositions wherein a ratio by weight of the gum to the RuBisCO protein isolate is about 1:40 to 1:50. Further provided here are compositions wherein the gum is xanthan gum. Further provided here are compositions wherein the composition when heated to gelation comprises water holding activity up to about 9 g water per g protein. Further provided here are compositions wherein the composition when heated to gelation comprises a cooked gel strength of up to about 12,500 g. Further provided here are compositions wherein the aqueous solution is water. Further provided here are compositions wherein the aqueous solution comprises water and one or more acids. Further provided here are compositions wherein the one or more acids comprises citric acid and/or ascorbic acid. Further provided here are compositions wherein the composition comprises: RuBisCO protein isolate, xanthan gum, ascorbic acid, citric acid, and water. Further provided here are compositions wherein the: RuBisCO protein isolate is present in an amount of about 11% by weight, the xanthan gum is present in an amount of about 0.25% by weight, the ascorbic acid is present in an amount of about 0.6% by weight, the citric acid is present in an amount of about 0.6% by weight, and the water is present in an amount of about 88% by weight. Further provided here are compositions wherein the composition comprises: about 5-15 weight % RuBisCO protein isolate, about 0.1-0.9 weight % of xanthan gum, and about 85-95 weight % of water. Further provided here are compositions wherein the RuBisCO protein isolate is free of chlorophyll. Further provided here are compositions wherein the RuBisCO protein isolate is flavorless and colorless. Further provided here are compositions wherein the RuBisCO protein isolate comprises a large subunit and a small subunit of RuBisCO protein. Further provided here are compositions wherein the RuBisCO protein isolate comprises protein comprising a sequence at least 90% identical to any one of SEQ ID NO: 1 to SEQ ID NO: 10. Further provided here are compositions wherein the RuBisCO protein isolate comprises protein comprising a sequence at least 95% identical to any one of SEQ ID NO: 1 to SEQ ID NO: 10. Further provided here are compositions wherein the RuBisCO protein isolate comprises protein comprising a sequence of SEQ ID NO: 1 or 2; SEQ ID NO: 3 or 4; SEQ ID NO: 5 or 6; SEQ ID NO: 7 or 8; or SEQ ID NO: 9 or 10. Further provided here are compositions wherein the RuBisCO protein isolate comprises proteins comprising sequence of SEQ ID NO: 1 and 2; SEQ ID NO: 3 and 4; SEQ ID NO: 5 and 6; SEQ ID NO: 7 and 8; or SEQ ID NO: 9 and 10. Further provided here are compositions wherein the RuBisCO protein isolate comprises a RuBisCO protein large subunit. Further provided here are compositions wherein the RuBisCO protein isolate comprises a RuBisCO protein small subunit. Further provided here are compositions wherein the RuBisCO protein isolate is from a plant in the Lemna genus. Further provided here are compositions wherein the RuBisCO protein isolate is from a Lemna minor. Further provided here are compositions wherein the RuBisCO protein isolate is from a Lemna aequinoctialis, Lemna disperma, Lemna ecuadoriensis, Lemna gibba, Lemna japonica, Lemna minor, Lemna minuta, Lemna obscura, Lemna paucicostata, Lemna perpusilla, Lemna tenera, Lemna trisulca, Lemna turionifera, Lemna valdiviana, Lemna yungensis, Medicago sativa, Nicotiana sylvestris, Nicotiana tabacum, Spinacia oleracea, Beta vulgaris, Atriplex lentiformis, Pereskia aculeata, and Chlorella vulgaris. Further provided here are compositions wherein the composition comprises a pH up to about 7.8. Further provided here are compositions wherein the composition comprises a pH of 5 to 7.8. Further provided here are compositions wherein the composition comprises tryptophan amount increased compared to a similar mass of egg white. Further provided here are compositions wherein the composition further comprises a food additive. Further provided here are compositions wherein the food additive is selected from the group consisting of: a plasticizer, a sugar, a flavoring component, a coloring component, a fiber, a soluble salt, a starch, an acid, and a wax. Further provided here are compositions wherein the flavoring component comprises a turmeric component. Further provided here are compositions wherein the fiber is selected from the group consisting of: pectin, citrus fiber, and cellulose. Further provided here are compositions wherein the soluble salt is calcium lactate gluconate. Further provided here are compositions wherein the acid is selected from the group consisting of: ascorbic acid and citric acid. Further provided here are compositions wherein the wax is selected from the group consisting of: a naturally-derived wax and a synthetic wax. Further provided here are compositions wherein comprising an ingredient from an animal. Further provided here are compositions comprising a synthetic ingredient. Further provided here are compositions wherein the RuBisCO comprises up to 20% by weight of the composition. Further provided here are compositions wherein the RuBisCO comprises up to 10% by weight of the composition. Further provided here are compositions wherein the RuBisCO comprises up to 5% by weight of the composition. Further provided here are compositions wherein the RuBisCO comprises up to 1% by weight of the composition. Further provided here are compositions wherein the composition is an egg replacement food product. Further provided here are compositions wherein the composition is a milk replacement food product. Further provided here are compositions wherein the composition comprises: water, canola oil, RuBisCO protein isolate, beta carotene, sunflower lecithin, tapioca starch, and xanthan gum. Further provided here are compositions wherein the water in an amount of about 70% by weight, the Canola Oil in an amount of about 9.5% by weight, the RuBisCO protein isolate in an amount of about 11% by weight, the tapioca starch in an amount of about 8% by weight, and the following in an amount of less than 1% by weight: beta carotene, sunflower lecithin, and xanthan gum. Further provided here are compositions further comprising sodium hydroxide. Further provided here are compositions wherein about 10-15 weight % RuBisCO protein isolate, about 10-15 weight % safflower oil, about 2-15 weight % coconut oil, about 2-15 weight % turmeric, about 0.1-10 weight % pectin, about 1-7 weight % calcium lactate gluconate, and about 50-65 weight % water. Further provided here are compositions wherein about 5-15 weight % RuBisCO protein isolate, about 2-10 weight 5% grapeseed oil, about 1-10 weight % coconut oil, about 0.1-0.9 weight % gellan gum, about 0.1-0.9 weight % calcium lactate gluconate, about 1-5 weight % tapioca starch, about 50.1-0.9 weight % cellulose, about 1-7 weight % turmeric, about 0.01-0.09 weight % ascorbic acid, about 0.01-0.09 weight % citric acid, and about 65-85 weight % water. Further provided here are compositions wherein about 5-15 weight % RuBisCO protein isolate, about 2-10 weight % grapeseed oil, about 2-7 weight % coconut oil, about 0.1-0.9 weight % gellan gum, about 0.1-0.9 weight % calcium lactate gluconate, about 1-5 weight % tapioca starch, about 0.1-0.9 weight % cellulose, about 1-5 weight % turmeric, about 65-85 weight % water, 25 about 0.01-0.09 weight % ascorbic acid, and about 0.01-0.09 weight % citric acid. Further provided here are compositions wherein about 5-15 weight % RuBisCO protein isolate, about 10-20 weight % safflower oil, about 0.1-0.9 weight % gellan gum, about 1-10 weight % tapioca starch, about 0.1-0.9 weight % pectin, about 0.1-0.9 weight % xanthan gum, about 1-4 weight % 5 turmeric, about 65-85 weight % water. Further provided here are compositions wherein: about 5-15 weight % RuBisCO protein isolate, about 5-15 weight % grapeseed oil, about 0.1-0.9 weight % locust bean gum, about 0.1-0.9 weight % xanthan gum, and about 65-85 weight % water. Further provided here are compositions wherein about 5-15 weight % RuBisCO protein isolate, about 5-15 weight % safflower oil, about 0.01-0.10 weight % beta carotene, about 65-85 weight % water, about 1-5 weight % baking powder, and about 0.1-0.9 weight % Givaudan Masker. Further provided here are compositions wherein about 1-5 weight % RuBisCO protein isolate, about 0.1-0.7 weight % guar gum, about 0.1-0.7 weight % sugar, about 1-5 weight % grapeseed oil, about 0.1-0.7 weight % natural flavoring, and about 85-95 weight % water. Further provided here are compositions wherein about 1-5 weight %, 5 RuBisCO protein isolate, about 1-5 weight % safflower oil, about 0.1-3 weight % guar gum, about 0.1-3 weight % citrus fiber, about 0.1-0.5 weight % natural flavor, and about 90-99 weight % water. Further provided here are compositions wherein about 1-5 weight %, RuBisCO protein isolate, about 1-5 weight % grapeseed oil, about 0.1-3 weight % guar gum, about 0.1-0.9 weight % sugar, about 0.1-0.9 weight % natural flavoring, and 20 about 90-99 weight % water. Further provided here are compositions wherein about 0.5-1.5, 15 weight % RuBisCO protein isolate, about 1-5 weight % safflower oil, about 0.1-0.9 weight % guar gum, about 0.1-0.5 weight % citrus fiber, and about 85-99 weight % water. Further provided here are compositions wherein about 0.5-1.5, weight % RuBisCO protein isolate, about 1-5 weight % grapeseed oil, about 0.1-0.9, 30 weight % guar gum, about 0.1-0.9 weight % sugar, about 0.1-0.9 weight % natural flavoring, and about 85-95 weight % water. Further provided here are compositions wherein about 1.5-3 weight % RuBisCO protein isolate, about 1-5 weight % grapeseed oil, about 0.1-0.9 weight % guar gum, about 0.1-0.9 weight % sugar, about 0.1-0.9 weight % natural flavoring, and about 85-95 weight % water. Further provided here are compositions wherein about 2-3 weight % RuBisCO protein isolate, about 30-40 weight % grapeseed oil, about 0.1-0.9 weight % guar gum, about 0.1-0.9 weight % xanthan gum, about 1-3 weight % sugar, about 1-3 weight % natural flavoring, and about 50-65 weight % water. Further provided here are compositions wherein about 4-7 weight % RuBisCO protein isolate, about 15-25 weight % safflower oil, about 10-20 weight % coconut oil, about 0.1-0.9 weight % guar gum, about 1-3 weight % natural flavoring, and about 50-65 weight % water. Further provided here are compositions wherein 4-7 weight % RuBisCO protein isolate, about 15-25 weight % safflower oil, about 10-20 weight 5% coconut oil, about 0.1-0.9 weight % guar gum, about 1-3 weight % natural flavoring, and about 50-65 weight % water. Further provided here are compositions wherein the composition is in a liquid, semisolid, or solid form.

Provided herein are compositions where the compositions comprise: a ribulose-1,5-bisphosphate carboxylase/oxygenase (RuBisCO) protein isolate, wherein the RuBisCO protein isolate is from a floating aquatic plant; and an aqueous solution, wherein a ratio by weight of the RuBisCO protein isolate to the aqueous solution is 1:4 to 1:100, or wherein the aqueous solution is present in an amount of at least 50% by weight. Further provided herein are compositions wherein a ratio by weight of the RuBisCO protein isolate to the aqueous solution is 1:4 to 1:20. Further provided herein are compositions wherein a ratio by weight of the RuBisCO protein isolate to the aqueous solution is 1:5 to 1:8. Further provided herein are compositions wherein a ratio by weight of the RuBisCO protein isolate to the aqueous solution is 1:8 to 1:20. Further provided herein are compositions wherein aqueous solution is present in an amount of at least 65% by weight. Further provided herein are compositions wherein the aqueous solution is present in an amount of at least 85% by weight. Further provided herein are compositions wherein the aqueous solution is present in an amount of up to 99% by weight. Further provided herein are compositions wherein the composition has a foaming capacity within about 5% of that of egg white when measured by percent overrun. Further provided herein are compositions wherein the composition initiates gelation when heated at a lower temperature than that of egg white. Further provided herein are compositions wherein the aqueous solution is water. Further provided herein are compositions wherein the aqueous solution comprises water and one or more acids. Further provided herein are compositions wherein the one or more acids comprises citric acid and/or ascorbic acid. Further provided herein are compositions wherein the RuBisCO protein isolate is free of chlorophyll. Further provided herein are compositions wherein the RuBisCO protein isolate is flavorless and colorless. Further provided herein are compositions wherein the RuBisCO protein isolate comprises a large subunit and a small subunit of RuBisCO protein. Further provided herein are compositions wherein the RuBisCO protein isolate comprises protein comprising a sequence at least 90% identical to SEQ ID NO: 1 or SEQ ID NO: 2. Further provided herein are compositions wherein the RuBisCO protein isolate comprises protein comprising a sequence at least 95% identical to SEQ ID NO: 1 or SEQ ID NO: 2. Further provided herein are compositions wherein the RuBisCO protein isolate comprises protein comprising a sequence of SEQ ID NO: 1 or SEQ ID NO: 2. Further provided herein are compositions wherein the RuBisCO protein isolate comprises proteins comprising sequence of SEQ ID NO: 1 and SEQ ID NO: 2. Further provided herein are compositions wherein the RuBisCO protein isolate comprises protein a RuBisCO protein large subunit. Further provided herein are compositions wherein the RuBisCO protein isolate comprises protein a RuBisCO protein small subunit. Further provided herein are compositions wherein the RuBisCO protein isolate is from a plant in the Lemna genus. Further provided herein are compositions wherein the RuBisCO protein isolate is from a Lemna minor. Further provided herein are compositions wherein the RuBisCO protein isolate is from a Lemna aequinoctialis, Lemna disperma, Lemna ecuadoriensis, Lemna gibba, Lemna japonica, Lemna minor, Lemna minuta, Lemna obscura, Lemna paucicostata, Lemna perpusilla, Lemna tenera, Lemna trisulca, Lemna turionifera, Lemna valdiviana, Lemna yungensis, Medicago sativa, Nicotiana sylvestris, Nicotiana tabacum, Spinacia oleracea, Beta vulgaris, Atriplex lentiformis, Pereskia aculeata, and Chlorella vulgaris. Further provided herein are compositions wherein the composition comprises a pH up to about 7.8. Further provided herein are compositions wherein the composition comprises a pH of 5 to 7.8. Further provided herein are compositions wherein the composition comprises tryptophan amount increased compared to a similar mass of egg white. Further provided herein are compositions wherein the composition further comprises a food additive. Further provided herein are compositions wherein the food additive is selected from the group consisting of: a plasticizer, a sugar, a flavoring component, a coloring component, a fiber, a soluble salt, a starch, an acid, and a wax. Further provided herein are compositions wherein the flavoring component comprises a turmeric component. Further provided herein are compositions wherein the fiber is selected from the group consisting of: pectin, citrus fiber, and cellulose. Further provided herein are compositions wherein the soluble salt is calcium lactate gluconate. Further provided herein are compositions wherein the acid is selected from the group consisting of: ascorbic acid and citric acid. Further provided herein are compositions wherein the wax is selected from the group consisting of: a naturally-derived wax and a synthetic wax. Further provided herein are compositions further comprising: an aqueous polysaccharide solution, an alcohol, a polyalcohol, a glycerol (glycerine), a gum Arabic, a xanthan gum, a guar gum, a locust bean gum, or an aqueous solution of carbohydrates. Further provided herein are compositions further comprising an ingredient from an animal. Further provided herein are compositions further comprising a synthetic ingredient. Further provided herein are compositions wherein the RuBisCO protein isolate comprises up to 20% by weight of the composition. Further provided herein are compositions wherein the RuBisCO protein isolate comprises up to 10% by weight of the composition. Further provided herein are compositions wherein the RuBisCO protein isolate comprises up to 5% by weight of the composition. Further provided herein are compositions wherein the RuBisCO protein isolate comprises up to 1% by weight of the composition. Further provided herein are compositions wherein the composition is an egg replacement food product. Further provided herein are compositions wherein the composition is a milk replacement food product. Further provided herein are compositions wherein the composition is in a liquid form, a semisolid form, or a solid form. Further provided herein are compositions wherein the compositions comprise: a ribulose-1,5-bisphosphate carboxylase/oxygenase (RuBisCO) protein isolate; and an oil; wherein a ratio by weight of the RuBisCO protein isolate to the oil is 10:1 to 1:20; or wherein the oil is present in amount of up to about 20% by weight. Further provided herein are compositions wherein the oil is present in amount of up to about 15% by weight. Further provided herein are compositions wherein the oil is present in amount of up to about 10% by weight. Further provided herein are compositions wherein the oil is present in amount of up to about 5% by weight. Further provided herein are compositions wherein the composition comprises oil holding capacity greater than egg white when measured by a ratio of grams of oil to grams of protein. Further provided herein are compositions wherein the composition is in a liquid form. Further provided herein are compositions wherein the composition is in a semisolid form. Further provided herein are compositions wherein the oil is plant-based. Further provided herein are compositions wherein the oil is animal-based. Further provided herein are compositions wherein the oil is synthetic. Further provided herein are compositions wherein the RuBisCO protein isolate is present in an amount of up to about 15% by weight of the composition. Further provided herein are compositions wherein the RuBisCO protein isolate is free of chlorophyll. Further provided herein are compositions wherein the RuBisCO protein isolate is flavorless and colorless. Further provided herein are compositions wherein the RuBisCO protein isolate comprises a large subunit and a small subunit of RuBisCO protein. Further provided herein are compositions wherein the RuBisCO protein isolate comprises protein comprising a sequence at least 90% identical to any one of SEQ ID NO: 1 to SEQ ID NO: 10. Further provided herein are compositions wherein the RuBisCO protein isolate comprises protein comprising a sequence at least 95% identical to any one of SEQ ID NO: 1 to SEQ ID NO: 10. Further provided herein are compositions wherein the RuBisCO protein isolate comprises protein comprising a sequence of SEQ ID NO: 1 or 2; SEQ ID NO: 3 or 4; SEQ ID NO: 5 or 6; SEQ ID NO: 7 or 8; or SEQ ID NO: 9 or 10. Further provided herein are compositions wherein the RuBisCO protein isolate comprises proteins comprising sequence of SEQ ID NO: 1 and 2; SEQ ID NO: 3 and 4; SEQ ID NO: 5 and 6; SEQ ID NO: 7 and 8; or SEQ ID NO: 9 and 10. Further provided herein are compositions wherein the RuBisCO protein isolate comprises protein comprising a RuBisCO protein large subunit. Further provided herein are compositions wherein the RuBisCO protein isolate comprises protein comprising a RuBisCO protein small subunit. Further provided herein are compositions wherein the RuBisCO protein isolate is from a plant in the Lemna genus. Further provided herein are compositions wherein the RuBisCO protein isolate is from a Lemna minor. Further provided herein are compositions wherein the RuBisCO protein isolate is from a Lemna aequinoctialis, Lemna disperma, Lemna ecuadoriensis, Lemna gibba, Lemna japonica, Lemna minor, Lemna minuta, Lemna obscura, Lemna paucicostata, Lemna perpusilla, Lemna tenera, Lemna trisulca, Lemna turionifera, Lemna valdiviana, Lemna yungensis, Medicago sativa, Nicotiana sylvestris, Nicotiana tabacum, Spinacia oleracea, Beta vulgaris, Atriplex lentiformis, Pereskia aculeata, and Chlorella vulgaris. Further provided herein are compositions further comprising an aqueous solution. Further provided herein are compositions wherein the aqueous solution is water. Further provided herein are compositions wherein the composition comprises emulsion activity greater than egg white when measured by absorbance of 500 nm light wavelength. Further provided herein are compositions wherein the composition comprises a pH up to about 7.8. Further provided herein are compositions wherein the composition comprises a pH of 5 to 7.8. Further provided herein are compositions wherein the composition comprises tryptophan amount increased compared to a similar mass of egg white. Further provided herein are compositions wherein the composition further comprises a food additive. Further provided herein are compositions wherein the food additive is selected from the group consisting of: a plasticizer, a sugar, a flavoring component, a coloring component, a fiber, a soluble salt, a starch, an acid, and a wax. Further provided herein are compositions wherein the flavoring component comprises a turmeric component. Further provided herein are compositions wherein the fiber is selected from the group consisting of: pectin, citrus fiber, and cellulose. Further provided herein are compositions wherein the soluble salt is calcium lactate gluconate. Further provided herein are compositions wherein the acid is selected from the group consisting of: ascorbic acid and citric acid. Further provided herein are compositions wherein the wax is selected from the group consisting of: a naturally-derived wax and a synthetic wax. Further provided herein are compositions wherein the oil is selected from the group consisting of: a safflower oil, a coconut oil, a grapeseed oil, and a canola oil. Further provided herein are compositions further comprising: an aqueous polysaccharide solution, an alcohol, a polyalcohol, a glycerol (glycerine), a gum Arabic, a xanthan gum, a guar gum, a locust bean gum, or an aqueous solution of carbohydrates. Further provided herein are compositions further comprising an ingredient from an animal. Further provided herein are compositions further comprising a synthetic ingredient. Further provided herein are compositions wherein the composition is lyophilized. Further provided herein are compositions wherein the composition is an egg replacement food product. Further provided herein are compositions wherein the composition is a milk replacement food product.

Provided herein are compositions, wherein the compositions comprise: a ribulose-1,5-bisphosphate carboxylase/oxygenase (RuBisCO) protein isolate, wherein the RuBisCO protein isolate is present in an amount of at least 70% by weight of the composition; a lipid; and a thickener, wherein the composition is in the form of a powder. Further provided herein are compositions wherein the powder is lyophilized. Further provided herein are compositions wherein the RuBisCO protein isolate is present in an amount of at least 80% by weight of the composition. Further provided herein are compositions wherein the RuBisCO protein isolate is free of chlorophyll. Further provided herein are compositions wherein the RuBisCO protein isolate is flavorless and colorless. Further provided herein are compositions wherein the RuBisCO protein isolate comprises a large subunit and a small subunit of RuBisCO protein. Further provided herein are compositions wherein the RuBisCO protein isolate comprises protein comprising a sequence at least 90% identical to any one of SEQ ID NO: 1 to SEQ ID NO: 10. Further provided herein are compositions wherein the RuBisCO protein isolate comprises protein comprising a sequence at least 95% identical to any one of SEQ ID NO: 1 to SEQ ID NO: 10. Further provided herein are compositions wherein the RuBisCO protein isolate comprises protein comprising a sequence of SEQ ID NO: 1 or 2; SEQ ID NO: 3 or 4; SEQ ID NO: 5 or 6; SEQ ID NO: 7 or 8; or SEQ ID NO: 9 or 10. Further provided herein are compositions wherein the RuBisCO protein isolate comprises proteins comprising sequence of SEQ ID NO: 1 and 2; SEQ ID NO: 3 and 4; SEQ ID NO: 5 and 6; SEQ ID NO: 7 and 8; or SEQ ID NO: 9 and 10. Further provided herein are compositions wherein the RuBisCO protein isolate comprises protein a RuBisCO protein large subunit. Further provided herein are compositions wherein the RuBisCO protein isolate comprises protein a RuBisCO protein small subunit. Further provided herein are compositions wherein the RuBisCO protein isolate is from a plant in the Lemna genus. Further provided herein are compositions wherein the RuBisCO protein isolate is from a Lemna minor. Further provided herein are compositions wherein the RuBisCO protein isolate is from a Lemna aequinoctialis, Lemna disperma, Lemna ecuadoriensis, Lemna gibba, Lemna japonica, Lemna minor, Lemna minuta, Lemna obscura, Lemna paucicostata, Lemna perpusilla, Lemna tenera, Lemna trisulca, Lemna turionifera, Lemna valdiviana, Lemna yungensis, Medicago sativa, Nicotiana sylvestris, Nicotiana tabacum, Spinacia oleracea, Beta vulgaris, Atriplex lentiformis, Pereskia aculeata, and Chlorella vulgaris. Further provided herein are compositions wherein the composition comprises a pH up to about 7.8. Further provided herein are compositions wherein the composition comprises a pH of 5 to 7.8. Further provided herein are compositions wherein the composition comprises tryptophan amount increased compared to a similar mass of egg white. Further provided herein are compositions wherein the composition further comprises a food additive. Further provided herein are compositions wherein the food additive is selected from the group consisting of: a plasticizer, a sugar, an oil, a flavoring component, a coloring component, a starch, and an acid. Further provided herein are compositions wherein the flavoring component comprises a turmeric component. Further provided herein are compositions wherein the acid is selected from the group consisting of: ascorbic acid and citric acid. Further provided herein are compositions further comprising: an aqueous polysaccharide solution, an alcohol, a polyalcohol, a glycerol (glycerine), a gum Arabic, a xanthan gum, a guar gum, a locust bean gum, or an aqueous solution of carbohydrates. Further provided herein are compositions further comprising an ingredient from an animal. Further provided herein are compositions further comprising a synthetic ingredient. Further provided herein are compositions wherein the RuBisCO protein isolate comprises up to 20% by weight of the composition. Further provided herein are compositions wherein the RuBisCO protein isolate comprises up to 10% by weight of the composition. Further provided herein are compositions wherein the RuBisCO protein isolate comprises up to 5% by weight of the composition. Further provided herein are compositions wherein the RuBisCO protein isolate comprises up to 1% by weight of the composition. Further provided herein are compositions wherein the composition is an egg replacement food product. Further provided herein are compositions wherein the composition is a milk replacement food product.

Provided herein are methods of manufacturing, comprising: spray drying or freeze drying the composition described herein. Provided herein are methods of manufacturing, comprising: providing a ribulose-1,5-bisphosphate carboxylase/oxygenase (RuBisCO) protein isolate, and mixing the RuBisCO with at least one of the following: an oil; wherein a ratio by weight of the RuBisCO protein isolate to the oil is 10:1 to 1:20; or wherein the oil is present in amount of up to about 20% by weight; a plasticizer, wherein a ratio by weight of the plasticizer to the RuBisCO protein isolate is about 1:5 to 1:200, or wherein the plasticizer is present in amount of up to about 8% by weight; and an aqueous solution, wherein a ratio by weight of the RuBisCO protein isolate to the aqueous solution is 1:4 to 1:100, or wherein the aqueous solution is present in an amount of at least 50% by weight.

Provided herein are plant-based food products comprising: a protein isolate; a food component; and a food additive. Further provided herein are plant-based food products wherein the food additive is selected from the group consisting of: a plasticizer, an oil, a sugar, a flavoring component, a coloring component, a fiber, a soluble salt, a starch, an acid, and a wax. Further provided herein are plant-based food products wherein the plasticizer is selected from the group consisting of: water, an aqueous polysaccharide solutions, an alcohol, a polyalcohol, a glycerol (glycerine), a gum Arabic, a xanthan gum, a guar gum, a locust bean gum, and an aqueous solution of carbohydrates. Further provided herein are plant-based food products wherein the coloring component comprises a turmeric component. Further provided herein are plant-based food products wherein the fiber is selected from the group consisting of: pectin, citrus fiber, and cellulose. Further provided herein are plant-based food products wherein the soluble salt is calcium lactate gluconate. Further provided herein are plant-based food products wherein the acid is selected from the group consisting of: ascorbic acid and citric acid. Further provided herein are plant-based food products wherein the oil is selected from the group consisting of: a safflower oil, a coconut oil, a grapeseed oil, and a canola oil. Further provided herein are plant-based food products wherein the wax is selected from the group consisting of: a naturally-derived wax and a synthetic wax. Further provided herein are plant-based food products wherein the protein isolate comprises a ribulose-1,5-bisphosphate carboxylase/oxygenase (RuBisCo) protein isolate. Further provided herein are plant-based food products wherein the RuBisCo protein isolate comprises a protein content greater than approximately 80%. Further provided herein are plant-based food products wherein the RuBisCo protein isolate is free of chlorophyll. Further provided herein are plant-based food products wherein the RuBisCo protein isolate is flavorless and colorless. Further provided herein are plant-based food products wherein the plant-based food product comprises a milk replacement product or an egg replacement product. Further provided herein are plant-based food products wherein the milk replacement product or the egg replacement product is used to create another food product.

### Examples

### Example 1—Egg White Replacement Product Formulation

In Example 1, an egg white replacement product formulation is described that includes approximately 5-15 wt. % of the RuBisCO protein isolate, approximately 0.1-0.9 wt. % of xanthan gum, and 85-95 wt. % of water. The RuBisCO protein isolate, the xanthan gum, and the water are mixed together thoroughly with a hand mixer until dissolved. The egg white replacement product of Example 1 can be cooked similarly to a normal egg white in a pan.

The resulting RuBisCO-based egg white has a cook texture, juiciness, and color similar to resuspended and cooked chicken egg white protein. The flavor of the RuBisCO-based egg white had a neutral protein taste with notes of sulfur. Rheological data indicated that the elastic modulus of the RuBisCO-based egg white was as strong or stronger than chicken egg white protein.

Specific data associated with this example is shown in TABLE 3 below.

TABLE 3

| | Water Holding | | | |
|---|---|---|---|---|
| | Capacity of Gel | Cooked Gel | | Solution |
| | (g water/g | Strength | | Viscometry |
| | protein) | (g) | pH | (cP) |
| Animal Egg White | 8.9 | 11489 | 8.8 | 1.7 |
| RuBisCO Egg White | 8.84 | 11745 | 7.4 | 5.6 |

FIG. 2 and FIG. 3 depict graphical plots showing gelation rheology results and modulus comparisons of RuBisCO protein isolate samples as compared to an egg white protein (EWP) sample, according to at least some embodiments disclosed herein.

It should be appreciated that a sample rheology protocol includes using a Bohlin CVO rheometer with a Peltier temperature control unit, a 20 mm parallel plate, and a constant frequency-strain oscillatory temperature sweep program, with a frequency of 0.1 Hz, a strain of 0.01, a gap of 500 μm, a cap temperature compensation of 3 μm 1° C., a heating rate of 10° C./minute and a heating profile as follows: about 7 minutes for 25-90° C., about 1 minute for 90-90° C., about 7 minutes for 90-25° C., and about 1 minute for 25-25° C.

The solutions may be prepared from chicken animal egg or RuBisCO egg mixes. In examples, approximately 1 mL was loaded onto the plate, the plate was lowered to the gap, and the edge was sealed with a thin layer of canola oil. Furthermore, the solvent trap was placed over the plate with water on the edges to maintain a high moisture environment. It should be appreciated that this sample rheology protocol is being provided for illustrative purposes only.

Specifically, FIG. 2 includes an x-axis 102 representing time in seconds and a y-axis 106 depicting an elastic modulus (in Pa) in view of varying temperatures 104 (in degrees Celsius). An elastic modulus is a quantity that measures an object or substance's resistance to being deformed elastically when a stress is applied to it. The elastic modulus of an object is defined as the slope of its stress—strain curve in the elastic deformation region. A stiffer material will have a higher elastic modulus.

An egg white protein sample 108, a first RuBisCO protein isolate sample 110, a second RuBisCO protein isolate sample 112, a third RuBisCO protein isolate sample 114, a fourth RuBisCO protein isolate sample 116, and a temperature 118 are plotted in FIG. 2. Each RuBisCO protein isolate sample and the egg white protein sample 108 each comprise a 10 wt. % solution concentration. The results depict that gels made from RuBisCO protein isolate samples are stronger than those made from the egg white protein sample 108.

FIG. 3 depicts a bar chart of the egg white protein sample 108, the first RuBisCO protein isolate sample 110, the second RuBisCO protein isolate sample 112, the third RuBisCO protein isolate sample 114, and the fourth RuBisCO protein isolate sample 116. The y-axis 106 of FIG. 3 measures the terminal elastic modulus (in Pa). The gelation behavior is similar among these samples, but the RuBisCO protein isolate samples begin gelation at a lower temperature (e.g., 65° C.), as compared to 75° C. for the egg white protein sample.

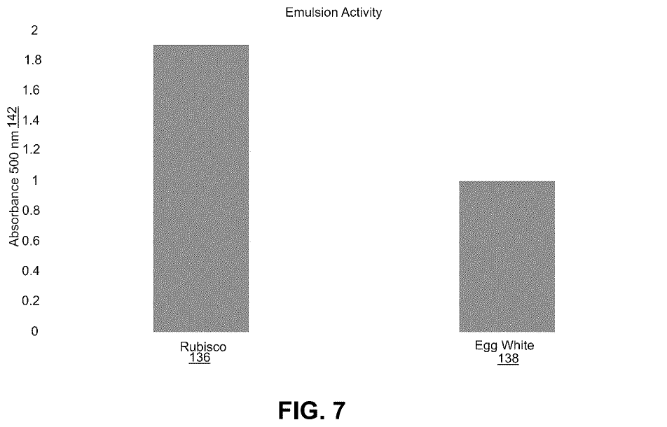

FIG. 7 depicts a graphical plot of an emulsion activity of a RuBisCo protein isolate and an emulsion activity of an egg white, according to at least some embodiments disclosed herein. FIG. 7 plots the emulsion activity 142 (measured in absorbance at 500 nm) of both a RuBisCo protein isolate 136 and an egg white 138. As shown in FIG. 7, the amount of emulsion activity 142 for the RuBisCo protein isolate 136 is greater than the amount of emulsion activity 142 for the egg white 138.

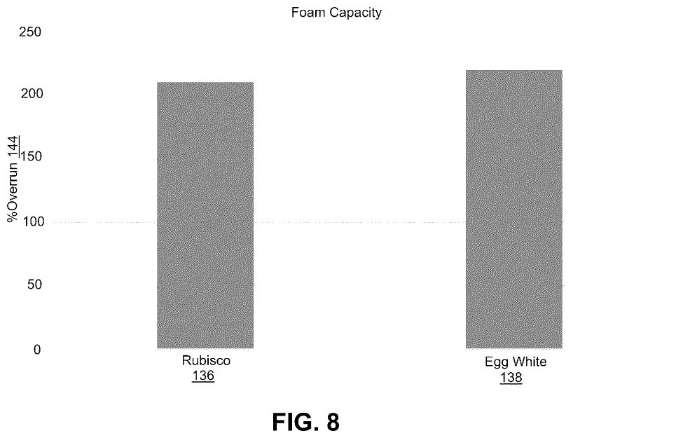

FIG. 8 depicts a graphical plot of a foam capacity of a RuBisCo protein isolate and a foam capacity of an egg white, according to at least some embodiments disclosed herein. FIG. 8 plots the foam capacity 144 (measured in % overrun) of both a RuBisCo protein isolate 136 and an egg white 138. As shown in FIG. 8, the foam capacity 144 is similar between the RuBisCo protein isolate 136 and the egg white 138.

### Example 2—Meringue Replacement Product Formulation

In Example 2, a meringue replacement product formulation is described that includes approximately 100-180 grams of the egg white replacement product of Example 1, approximately 120-140 grams of sugar, approximately 1.5-1.9 mg of salt, approximately 1-9 grams of vanilla flavoring, and approximately 2-6 grams of cream of tartar. The egg white replacement product of Example 1, the salt, and the cream of tartar were combined in a mixing bowl and placed into a stand mixer. The components were whipped using a whisk attachment on low until foaming begins. The speed was increased until a high speed was achieved, and the sugar was gradually added in the mixture until it was all added and dissolved. The mixing continued until the mixture was stiff and shiny. Next, the vanilla flavoring was added to the mixture. The mixture was then placed into a piping bag and piped out in approximately 1.5″ diameter circles onto parchment paper. The product was baked at approximately 225° F. for approximately 1.5 hours. The product was then cooled for approximately 6-8 hours.

The resulting RuBisCO-based meringue had a cooked color and shape that was identical to chicken egg-based meringue. The bite and chew of the RuBisCO-based meringue was identical to the chicken egg-based meringue and no off-flavor was detected in the RuBisCO-based meringue.

### Example 3—Egg Yolk Replacement Product Formulation

In Example 3, an egg yolk replacement product formulation is described that includes approximately 10-15 wt. % RuBisCO protein isolate, approximately 10-15 wt. % safflower oil, approximately 2-15 wt. % coconut oil, approximately 2-15 wt. % turmeric, approximately 0.1-10 wt. % pectin, approximately 1-7 wt. % calcium lactate gluconate, and approximately 50-65 wt. % water. All dry ingredients are weighted into a container and water is added to the container. The sample is mixed using a hand blender until the dry ingredients dissolve. The oil ingredients are added and the sample is homogenized thoroughly using a hand blender. The material is then placed into a mold and frozen.

An approximately 0.1-0.9 wt. % solution of alginate is made and heated to a temperature of approximately 130-140° F. Alginic acid is a polysaccharide distributed widely in the cell walls of brown algae that is hydrophilic and forms a viscous gum when hydrated. The frozen egg yolk product is placed into the alginate bath and is allowed to thaw and gel with the alginate for approximately 4-6 minutes. The egg yolk product is then removed from the alginate bath, strained, and rinsed. The egg yolk product may be cooked between approximately 2-6 minutes depending on the desired firmness.

The cooked egg yolk product exhibits viscosity variation, resulting in a runny solution to a firm gel depending on the cook time.

### Example 4—Egg Yolk Replacement Product Formulation

In Example 4, an egg yolk replacement product formulation is described that includes approximately 5-15 wt. % RuBisCO protein isolate, approximately 2-10 wt. % grapeseed oil, approximately 1-10 wt. % coconut oil, approximately 0.1-0.9 wt. % gellan gum, approximately 0.1-0.9 wt. % calcium lactate gluconate, approximately 1-5 wt. % tapioca starch, approximately 0.1-0.9 wt. % cellulose, approximately 1-7 wt. % turmeric, approximately 0.01-0.09 wt. % ascorbic acid, approximately 0.01-0.09 wt. % citric acid, and approximately 65-85 wt. % water. All dry ingredients are weighted into a container and water is added to the container. The sample is mixed using a hand blender until the dry ingredients dissolve. The oil ingredients are added and the sample is homogenized thoroughly using a hand blender. The material is then placed into a mold and frozen.

An approximately 0.1-0.9 wt. % solution of alginate is made and is heated to approximately 130-140° F. The frozen egg yolk product is placed into the alginate bath and allowed to thaw and gel with the alginate for approximately 4-6 minutes. The egg yolk product is then removed from the alginate bath, strained, and rinsed. The egg yolk product may also be cooked between approximately 2-6 minutes depending on the desired firmness.

The cooked egg yolk product exhibits viscosity variation, resulting in a runny solution to a firm gel depending on the cook time.

### Example 5—Whole Homogenized Egg Replacement Product Formulation

In Example 5, a whole homogenized egg replacement product is described that includes approximately 5-15 wt. % RuBisCO protein isolate, approximately 2-10 wt. % grapeseed oil, approximately 2-7 wt. % coconut oil, approximately 0.1-0.9 wt. % gellan gum, approximately 0.1-0.9 wt. % calcium lactate gluconate, approximately 1-5 wt. % tapioca starch, approximately 0.1-0.9 wt. % cellulose, approximately 1-5 wt. % turmeric, approximately 65-85 wt. % water, approximately 0.01-0.09 wt. % ascorbic acid, and approximately 0.01-0.09 wt. % citric acid.

All dry ingredients are weighed into a container and water is added to the container. The sample is mixed using a hand blender until the dry ingredients dissolve. The oil ingredients are added and the sample is homogenized thoroughly using a hand blender. The whole homogenized egg product can be cooked as an omelet or a scrambled preparation.

### Example 6—Whole Homogenized Egg Replacement Product Formulation

In Example 6, a whole homogenized egg replacement product is described that includes approximately 5-15 wt. % RuBisCO protein isolate, approximately 10-20 wt. % safflower oil, approximately 0.1-0.9 wt. % gellan gum, approximately 1-10 wt. % tapioca starch, approximately 0.1-0.9 wt. % pectin, approximately 0.1-0.9 wt. % xanthan gum, approximately 1-4 wt. % turmeric, approximately 65-85 wt. % water.

All dry ingredients are weighed into a container and water is added to the container. The sample is mixed using a hand blender until the dry ingredients dissolve. The oil ingredients are added and the sample is homogenized thoroughly using a hand blender. The pH of the final egg product is adjusted to pH 7-7.6. The whole homogenized egg product can be cooked as an omelet or a scrambled preparation.

The cooked egg product as a scramble has a bite and chew that are similar to scrambled chicken egg products. The flavor of the scrambled egg was comparable to chicken egg and the color was deemed acceptable in the range of colors exhibited by chicken-based eggs. The cooked egg product as an omelette was able to form a stable base layer that could be folded into a half-circle shape during cooking. The cooked texture had a bite, chew, and juiciness similar to a chicken egg omelette. Additionally, the smell and flavor of the omelette was similar to that of a chicken egg omelette.

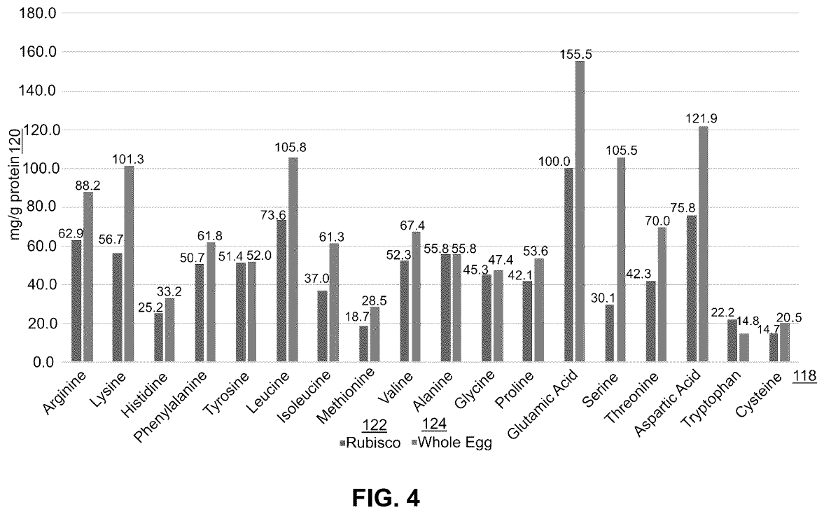

FIG. 4 depicts a graphical plot of an amino acid profile of a RuBisCO protein isolate and an amino acid profile of a whole egg, according to at least some embodiments disclosed herein. The graphical plot of FIG. 4 has an x-axis 118 associated with various amino acids (including arginine, lysine, histidine, etc.) and a y-axis 120 associated with measurements of the amino acids in mg/g protein for a RuBisCO protein isolate 122 and a whole egg 124. As shown in FIG. 4, the various amounts of amino acids are comparable between the whole egg 124 and the RuBisCO protein isolate 122.

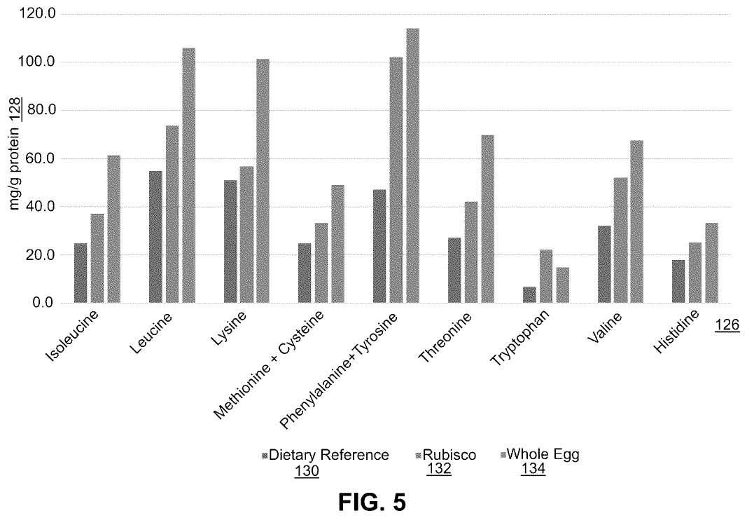

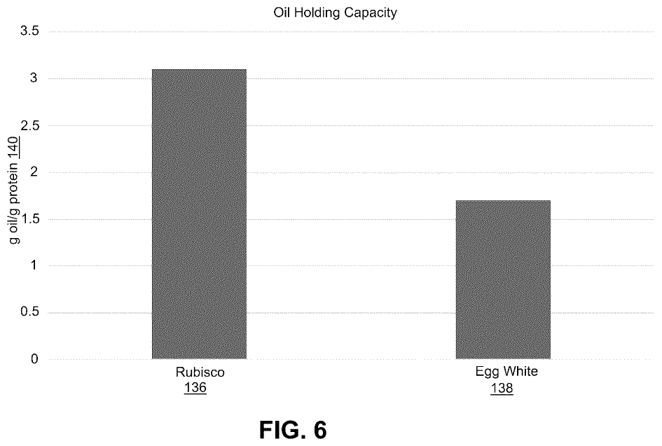

FIG. 5 depicts a graphical plot of essential amino acids for a dietary reference, a RuBisCO protein isolate, and a whole egg, according to at least some embodiments disclosed herein. FIG. 5 includes an x-axis 126 associated with various essential amino acids and a y-axis 128 associated with an amount of essential amino acids in mg/g protein for a dietary reference 130, a RuBisCO protein isolate 132, and a whole egg 134. It should be appreciated that the dietary reference 130 is based on the recommended levels for nutrition to be considered a complete protein. As shown in FIG. 5, the amounts of essential amino acids are generally highest for the whole egg 134, with the amounts of essential amino acids generally being second highest for the RuBisCO protein isolate 132.

Specific data associated with this example is shown in TABLE 4 below.

TABLE 4

| | Water Holding | | | |
|---|---|---|---|---|
| | Capacity of Gel | Cooked Gel | | Solution |
| | (g water/g | Strength | | Viscometry |
| | protein) | (g) | pH | (cP) |
| Animal Whole | 8.97 | 12751.15 | 7.5 | 18.2 |
| Homogenized Egg | | | | |
| RuBisCO Whole | 8.86 | 12538.85 | 6.8 | 25 |
| Homogenized Egg | | | | |

### Example 7—Egg Replacement Product Formulation for Baking

In Example 7, an egg replacement product formulation for baking is described that includes approximately 5-15 wt. % RuBisCO protein isolate, approximately 5-15 wt. % grapeseed oil, approximately 0.1-0.9 wt. % locust bean gum, approximately 0.1-0.9 wt. % xanthan gum, and approximately 65-85 wt. % water.

All dry ingredients are weighed into a container and water is added to the container. The sample is mixed using a hand blender until the dry ingredients dissolve. The oil ingredients are added and the sample is homogenized thoroughly using a hand blender. The egg replacement product can be used directly as a one to one replacement in baking recipes.

### Example 8—Egg Replacement Product Formulation for Baking

In Example 8, an egg replacement product formulation for baking is described that includes approximately 5-15 wt. % RuBisCO protein isolate, approximately 5-15 wt. % safflower oil, approximately 0.01-0.10 wt. % beta carotene, approximately 65-85 wt. % water, approximately 1-5 wt. % baking powder, and approximately 0.1-0.9 wt. % Givaudan Masker (natural flavor).

All dry ingredients are weighed into a container and water is added to the container. The sample is mixed using a hand blender until the dry ingredients dissolve. The oil ingredients are added and the sample is homogenized thoroughly using a hand blender. The egg replacement product can be used directly as a one to one replacement in baking recipes.

### Example 9—Milk Replacement Product Formulation

In Example 9, a milk replacement product is described that includes approximately 1-5 wt. % RuBisCO protein isolate, approximately 0.1-0.7 wt. % guar gum, approximately 0.1-0.7 wt. % sugar, approximately 1-5 wt. % grapeseed oil, approximately 0.1-0.7 wt. % natural flavoring, and approximately 85-95 wt. % water.

The resulting milk replacement has a creamy mouthfeel, white color, and flavor similar to cow whole milk.

### Example 10—Egg Replacement Product Formulation Used In Yellow Cake

In Example 10, the egg replacement product formulation (e.g., of Example 7 or Example 8) is used with a traditional yellow cake mix, canola oil, and the milk replacement product (e.g., of Example 9). Specifically, the components include 45-55 grams of the egg replacement product formulation of Example 7 or Example 8, 200-240 grams of cake mix, 35-50 mL canola oil, and 135-150 grams of the milk replacement product of Example 9. The cake mix, the oil, and the milk replacement product are added to a mixing bowl and mixed using a stand mixer with a whisk attachment at low speed for approximately 2 minutes. The egg replacement product formulation of Example 7 or Example 8 is added and the mixture is mixed for another 2 minutes. The mixture is baked at approximately 325° F. for approximately 18-20 minutes.

The cupcakes had a similar rise during baking as those cooked with animal egg and milk. The texture of the cupcakes was identical to those made with animal-based products. The cooked color, smell, and flavor of the cupcakes was also identical to those made with animal-based products.

### Example 11—Egg Replacement Product Formulation Used In Yellow Cake

The components of Example 11 include: approximately 250-350 grams of flour, approximately 5-15 grams of baking powder, approximately 0.1-1 grams of salt, approximately 225-325 grams of granulated sugar, approximately 150-200 grams of butter, approximately 5-15 mL of vanilla, approximately 100-200 grams of eggs or the egg replacement product formulation of Example 7 or Example 8, and approximately 250-350 mL of milk or the milk replacement product of Example 9.

The method to create the egg replacement product formulation used in yellow cake of Example 11 includes: mixing all of the dry ingredients together and cutting in shortening until the mixture resembles that of a wet or moist sand. Next, the eggs or the egg replacement product formulation of Example 7 or Example 8, the milk or the milk replacement product of Example 9, and the vanilla are added to the mixture. The mixture is then placed into a pan and is baked at 350° F. (176° C.) for a time period between 25-35 minutes.

Specific data associated with this example is shown in TABLE 5 and TABLE 6 below.

TABLE 5

| | Raw | Raw | | Raw | Raw | Cooked |
|---|---|---|---|---|---|---|
| | Viscometry | Density | Raw | Height | MC | Texture |
| Yellow Cake | (cP) | (g/cm³) | pH | (cm) | (%) | (g) |
| Ova Egg | 4433.33 | 1.00 | 7.35 | 3.6 | 33.89 | 306.0 |
| Ru BisCO Egg | 6000.00 | 0.96 | 7.69 | 3.6 | 31.49 | 289.12 |

TABLE 6

| | Cook | Cook | Cooked | Cook | | | |
|---|---|---|---|---|---|---|---|
| Yellow | Water | MC | Density | Height | Cooked | | Sen- |
| Cake | Loss (%) | (%) | (g/cm³) | (cm) | pH | Color | sory |
| Ova Egg | 9.54 | 30.63 | 1.09 | 5.2 | 6.69 | Same | Pass |
| RuBisCO | 9.39 | 35.24 | 1.14 | 5.5 | 6.64 | Same | Pass |
| Egg | | | | | | | |

### Example 12—Egg Replacement Product Formulation Used In Pound Cake

In Example 12, the egg replacement product formulation of Example 7 or Example 8 is used with butter, sugar, vanilla flavoring, flour, and salt. Specifically, the components of Example 12 include 150-250 grams of the egg replacement product formulation of Example 7 or Example 8, 120-135 grams of butter, 120-135 grams of sugar, 200-220 grams of flour, and 1-5 grams of salt. The butter, vanilla, and the sugar are creamed for approximately 7-8 minutes in a stand mixer with a whisk attachment. The egg replacement product formulation of Example 7 or Example 8 is mixed into the mixture slowly. The flour and salt are then added slowly to the mixture for approximately 1-2 minutes. The batter is baked for approximately 60-70 minutes at a temperature of approximately 325° F. (162° C.).

The cooked cake had a rise and crust color similar to that of animal-based pound cake. The chew and mouthfeel of the pound cake was identical to that of cake cooked with chicken eggs. There was also no difference in flavor, smell, or color in the RuBisCO-based pound cake.

Specific data regarding this example is shown in TABLE 7 and TABLE 8 below.

TABLE 7

| | Raw | | Cook Water | Cooked |
|---|---|---|---|---|
| | Viscometry | % Solids | Loss | Density |
| Pound Cake | (cP) | Raw/Cook | (%(g)) | (g/cm³) |
| Ova Egg | 8333.3 | 82.47/74.57 | 14.02 (83.65) | 0.638 |
| RuBisCO Egg | 80333.3 | 84.13/77.8 | 12.74 (75.88) | 0.657 |

TABLE 8

| | Cook Height | pH | | |
|---|---|---|---|---|
| Pound Cake | (cm) | Raw/Cook | Color | Sensory |
| Ova Egg | 8.08 | 7.1/7.2 | Same | Pass |
| RuBisCO Egg | 8.14 | 6.4/6.8 | Same | Pass |

### Example 13—Whole Milk Product Formulation

In Example 13, a whole milk product is described that includes approximately 1-5 wt. % RuBisCO protein isolate, approximately 1-5 wt. % safflower oil, approximately 0.1-3 wt. % guar gum, approximately 0.1-3 wt. % citrus fiber, approximately 0.1-0.5 wt. % natural flavor, and approximately 90-99 wt. % water.

All dry ingredients are weighed and placed into a container. Water is added to the container. The sample is mixed using a hand blender until the dry ingredients dissolve. The oil is added and the sample is homogenized thoroughly using a hand blender. The whole milk product can be drunk directly and it has a neutral taste and smell, an off-white color, and a smooth mouthfeel. The final milk product has a viscosity of 1.75-7 cP, a pH of 6.4-7, and an emulsion activity of 1.7-2. Bovine milk has a viscosity of 1.6-1.83, a pH of 6.6-7, and an emulsion activity of 2-2.3.

### Example 14—Whole Milk Product Formulation

In Example 14, a whole milk product is described that includes approximately 1-5 wt. % RuBisCO protein isolate, approximately 1-5 wt. % grapeseed oil, approximately 0.1-3 wt. % guar gum, approximately 0.1-0.9 wt. % sugar, approximately 0.1-0.9 wt. % natural flavoring, and approximately 90-99 wt. % water.

All dry ingredients are weighed and placed into a container. Water is added to the container. The sample is mixed using a hand blender until the dry ingredients dissolve. The oil is added and the sample is homogenized thoroughly using a hand blender. The whole milk product can be drunk directly and it has a neutral taste and smell, an off-white color, and a smooth mouthfeel. The final milk product has a viscosity of 1.75-7 cP, a pH of 6.4-7, and an emulsion activity of 1.7-2. Bovine milk has a viscosity of 1.6-1.83, a pH of 6.6-7, and an emulsion activity of 2-2.3.

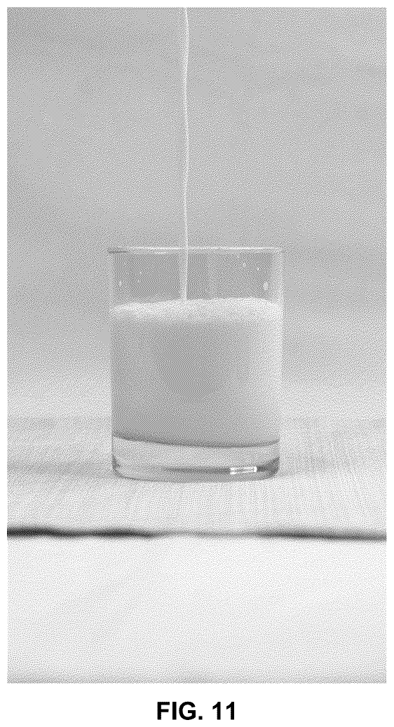

The milk formulation of Examples 13 and/or Example 14 is depicted in FIG. 11.

### Example 15—Cupcakes Using the Whole Milk Product Formulation of Example 14

In this example, cupcakes may be made using approximately 45-55 g RuBisCO egg replacer, approximately 300-400 g yellow cake mix, approximately 40-50 mL canola oil, and approximately 100-150 g of the whole milk product formulation of Example 14. The RuBisCO egg replacer includes approximately 10-19 wt. % RuBisCO protein isolate, approximately 2-11 wt. % grapeseed oil, approximately 0.1-0.9 wt. % locust bean gum, approximately 0.1-0.9 wt. % xanthan gum, and approximately 70-85 wt. % water.

The cake mix, the oil, and the RuBisCO whole milk product of Example 14 are added to a mixing bowl and beat using a stand mixer with whisk attachment at low speed for approximately 2 minutes. The RuBisCO egg replacer is added to the mixture and beat for approximately 2 more minutes. The mixture is then placed into cupcake holders and baked at approximately 325° F. (163° C.) for approximately 18-20 minutes.

### Example 16—Barista Milk Product Formulation

In Example 16, barista milk product formulation is made using approximately 0.5-1.5 wt. % RuBisCO protein isolate, approximately 1-5 wt. % safflower oil, approximately 0.1-0.9 wt. % guar gum, approximately 0.1-0.5 wt. % citrus fiber, and approximately 85-99 wt. % water.

All dry ingredients are weighed and placed into a container. Water is added to the container. The sample is mixed using a hand blender until the dry ingredients dissolve. The oil is then added and the sample is homogenized thoroughly using a hand blender.

In some examples, the barista milk product formulation of Example 16 may be added directly to hot black coffee and mixed. The resulting coffee had a cream color develop, a smooth texture, and a milder taste than black coffee.

The final milk product has a viscosity of 1.75-7 cP, a pH of 6.4-7, and an emulsion activity of 1.7-2. Bovine milk has a viscosity of 1.6-1.83, a pH of 6.6-7, and an emulsion activity of 2-2.3.

### Example 17—Barista Milk Product Formulation

In Example 17, the barista milk product formulation is made using approximately 0.5-1.5 wt. % RuBisCO protein isolate, approximately 1-5 wt. % grapeseed oil, approximately 0.1-0.9 wt. % guar gum, approximately 0.1-0.9 wt. % sugar, approximately 0.1-0.9 wt. % natural flavoring, and approximately 85-95 wt. % water.

All dry ingredients are weighed and placed into a container. Water is added to the container. The sample is mixed using a hand blender until the dry ingredients dissolve. The oil is then added and the sample is homogenized thoroughly using a hand blender.

In some examples, the barista milk product formulation of Example 17 may be added directly to hot black coffee and mixed. The resulting coffee had a cream color develop, a smooth texture, and a milder taste than black coffee. The final milk product has a viscosity of 1.75-7 cP, a pH of 6.4-7, and an emulsion activity of 1.7-2. Bovine milk has a viscosity of 1.6-1.83 cP, a pH of 6.6-7, and an emulsion activity of 2-2.3.

### Example 18—Barista Milk Product Formulation

In Example 18, heavy cream milk product formulation is made using approximately 1.5-3 wt. % RuBisCO protein isolate, approximately 1-5 wt. % grapeseed oil, approximately 0.1-0.9 wt. % guar gum, approximately 0.1-0.9 wt. % sugar, approximately 0.1-0.9 wt. % natural flavoring, and approximately 85-95 wt. % water.

All dry ingredients are weighed and placed into a container. Water is added to the container. The sample is mixed using a hand blender until the dry ingredients dissolve. The oil is then added and the sample is homogenized thoroughly using a hand blender.

The heavy cream product can be drunk directly or used in cream applications. It has a neutral taste and smell, an off-white color, and a smooth mouthfeel. The heavy cream product has a viscosity of 22-30 cP, a pH of 6.9-7, and an emulsion activity of 1.6-1.9. Bovine heavy cream has a viscosity of 22-28 cP, a pH of 6.9-7, and an emulsion activity of 3.2-3.8.

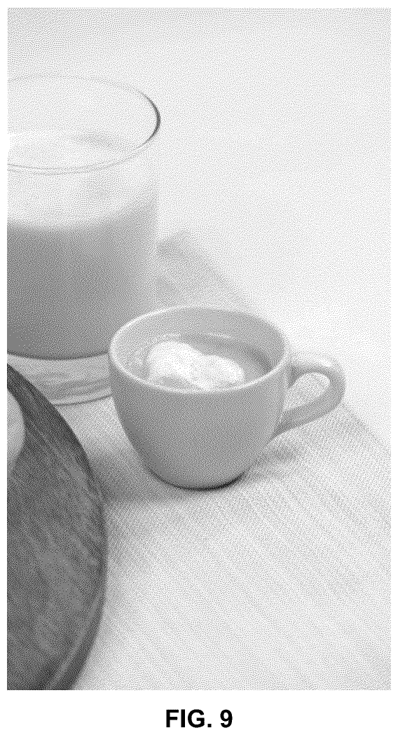

The barista milk product formulation of Examples 16, 17, and/or 18 is depicted in FIG. 9.

### Example 19—Custard Product Formulation

In Example 19, a custard was formed from approximately 2-3 wt. % RuBisCO protein isolate, approximately 30-40 wt. % grapeseed oil, approximately 0.1-0.9 wt. % guar gum, approximately 0.1-0.9 wt. % xanthan gum, approximately 1-3 wt. % sugar, approximately 1-3 wt. % natural flavoring, and approximately 50-65 wt. % water.

All dry ingredients are weighed and placed into a container. Water is added to the container. The sample is mixed using a hand blender until the dry ingredients dissolve. The oil is then added and the sample is homogenized thoroughly using a hand blender. The sample may then be heated in a 70° C. water bath for approximately 5-15 minutes with stirring to develop a custard texture. The resulting warm custard had a smooth mouthfeel, good mouth coating, a mildly sweet texture, and an off-white color.

The custard mixture prior to heating had a viscosity of 1500-2100 cP and a pH of 6.3-6.9. After heating, the custard product had a viscosity of 2600-3200 cP and a pH of 6.3-6.8.

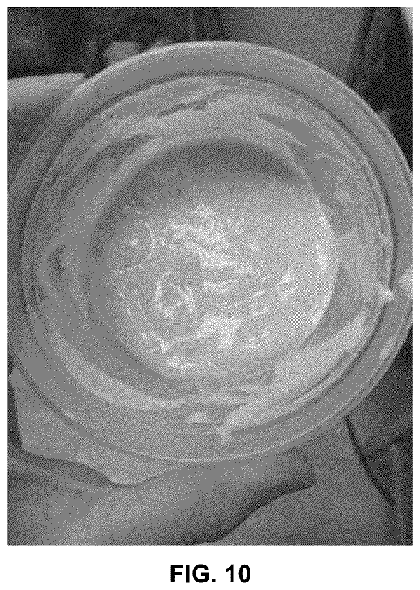

The custard product formulation of Example 19 is depicted in FIG. 10.

### Example 20—Yogurt

In Example 20, a yogurt was formed from approximately 4-7 wt. % RuBisCO protein isolate, approximately 15-25 wt. % safflower oil, approximately 10-20 wt. % coconut oil, approximately 0.1-0.9 wt. % guar gum, approximately 1-3 wt. % natural flavoring, and approximately 50-65 wt. % water.

All dry ingredients are weighed and placed into a container. Water is added to the container. The sample is mixed using a hand blender until the dry ingredients dissolve. The oil is then added and the sample is homogenized thoroughly using a hand blender. The sample may then be heated in a 70° C. water bath for approximately 20-25 minutes with stirring to develop a yogurt texture. The resulting yogurt was cooled to 4-8° C. prior to tasting. The resulting sour cream had a smooth mouthfeel, good mouth coating, and a white color.

The yogurt mixture prior to heating had a viscosity of 450-510 cP and a pH of 4.2-4.5. After heating, the yogurt product had a viscosity of 280-350 cP and a pH of 4.2-4.5. Bovine plain yogurt has a viscosity of 350-410 cP and a pH of 4.2-4.5.

### Example 21—Sour Cream

In Example 21, a sour cream was formed from approximately 4-7 wt. % RuBisCO protein isolate, approximately 15-25 wt. % safflower oil, approximately 10-20 wt. % coconut oil, approximately 0.1-0.9 wt. % guar gum, approximately 1-3 wt. % natural flavoring, and approximately 50-65 wt. % water.

All dry ingredients are weighed and placed into a container. Water is added to the container. The sample is mixed using a hand blender until the dry ingredients dissolve. The oil is then added and the sample is homogenized thoroughly using a hand blender. The sample may then be heated in a 70° C. water bath for approximately 20-25 minutes with stirring to develop a sour cream texture. The resulting sour cream was cooled to 4-8° C. prior to tasting. The resulting sour cream had a smooth mouthfeel, good mouth coating, and a white color.

The sour cream mixture prior to heating had a viscosity of 100-140 cP and a pH of 6.6-6.8. After heating, the yogurt product had a viscosity of 525-75000 cP and a pH of 6.6-6.8. Bovine sour cream has a viscosity of 75000-100000 cP and a pH of 4.2-4.5.

### Example 22—Sugar Cookies

The ingredients or components of this example include: approximately 400-500 grams of flour, approximately 1-10 grams of baking powder, approximately 1-5 grams of salt, approximately 150-250 grams of granulated sugar, approximately 200-300 grams of butter, and approximately 50-100 grams of eggs or the egg replacement product formulation of Example 7 or Example 8.

The method to create the sugar cookies includes: mixing all dry ingredients together and using a paddle attachment of a stand mixer to mix the butter and sugar for a time period of about 3-5 minutes. Next, the method includes adding the eggs or the egg replacement product formulation of Example 7 or Example 8 and the milk into the mixture and mixing the components at a medium speed for a time period of about 30 seconds. Next, the method includes adding the dry blend into the mixture and mixing the components until all ingredients are incorporated. The method then includes refrigerating the dough for a time period of about an hour and then rolling the dough into an approximately 8 mm thickness and cut into about 3⅜″ diameter circles. The sugar cookies are cooked at a temperature of about 375° F. (190° C.) for a time period of about 10 minutes.

Specific data regarding this example is shown in TABLE 9 and TABLE 10 below.

TABLE 9

| | Raw | Cook | Raw | Cook | Raw | Cooked |
|---|---|---|---|---|---|---|
| | Texture | Texture | MC | MC | Density | Density |
| Sugar Cookie | (g) | (g) | (%) | (%) | (g/mL) | (g/mL) |
| Ova Egg | 7870.3 | 11925.65 | 2.46 | 3.26 | 1.11 | 0.733 |
| RuBisCO Egg | 7766.5 | 11624.55 | 1.23 | 3.60 | 1.11 | 0.748 |

TABLE 10

| | | | | | | | Weight |
|---|---|---|---|---|---|---|---|
| | Raw | Cook | | | | | Loss after |
| Sugar | Height | Height | Raw | Cook | | | Cooking |
| Cookie | (mm) | (mm) | pH | pH | Color | Sensory | (g) |
| Ova Egg | 8 | 10.05 | 5.9 | 6.3 | Same | Same | 4.38 |
| RuBisCO | 8.2 | 11.5 | 5.7 | 6.2 | Same | Same | 3.33 |
| Egg | | | | | | | |

### Example 23—Muffins

The components of Example 23 include: approximately 200-300 grams of flour, approximately 50-150 grams of sugar, approximately 5-15 grams of baking powder, approximately 1-5 grams of salt, approximately 100-200 grams of butter, approximately 100-200 mL of milk or the whole milk product formulation of Examples 13 or 14, and approximately 75-125 grams of eggs or the egg replacement product formulation of Example 7 or Example 8.

The method to create the muffins includes whisking the flour, the sugar, the baking powder, and the salt to form a first mixture and whisking the milk or the whole milk product formulation of Examples 13 or 14, the butter, and the eggs or the egg replacement product formulation of Example 7 or Example 8 to form a second mixture. The method then includes combining the first mixture and the second mixture to form a third mixture. Next, the method includes baking the third mixture in muffin cups at a temperature of approximately 350° F. (176° C.) for a time period between 20-25 minutes.

Specific data regarding this example is shown in TABLE 11 and TABLE 12 below.

TABLE 11

| | Raw | Raw | | Raw | Raw | Cooked |
|---|---|---|---|---|---|---|
| | Viscometry | Density | Raw | Height | MC | Texture |
| Muffins | (cP) | (g/cm³) | pH | (cm) | (%) | (g) |
| Ova Egg | 9600.00 | 1.11 | 7.23 | 1.4 | 26.19 | 457.7 |
| RuBisCO | 8166.67 | 1.15 | 6.92 | 1.4 | 27.02 | 554.9 |
| Egg | | | | | | |

TABLE 12

| | Cook | Cooked | Cook | Cook | | | |
|---|---|---|---|---|---|---|---|
| | Water | Density | Height | MC | Cook | | |
| Muffins | Loss (%) | (g/cm³) | (cm) | (%) | pH | Color | Sensory |
| Ova Egg | 12.32 | 1.00 | 4.3 | 25.48 | 6.32 | Same | Pass |
| RuBisCO | 12.45 | 0.96 | 3.8 | 25.83 | 6.35 | Same | Pass |
| Egg | | | | | | | |

### Example 24—Donut

The components of Example 24 include: approximately 500-600 grams of flour, approximately 45-55 grams of sugar, approximately 2-10 grams of active yeast, approximately 75-125 grams of butter, approximately 200-300 mL of milk or the whole milk product formulation of Examples 13 or 14, approximately 1-5 grams of salt, and approximately 75-125 grams of eggs or the egg replacement product formulation of Example 7 or Example 8.

The method to create the donut of this example includes: heating the milk or the whole milk product formulation of Examples 13 or 14 until warm and combining the milk or the whole milk product formulation of Examples 13 or 14 with the yeast. Next, the method includes beating the eggs or the egg replacement product formulation of Example 7 or Example 8, the butter, the sugar, and the salt into the yeast and milk blend in a stand mixer with a dough hook. Then, the method includes adding half of the flour and beating until the components are mixed. The method then includes adding the second half of the flour until the dough pulls away from the mixing bowl. Next, the method includes rolling the dough onto a floured surface to approximately a′/2″ thickness and cutting the dough into a approximately 3″ circles. Then, the method includes allowing the dough to rise for a time period about 45 minutes. Next, the method includes heating frying oil to a temperature of about 375° F. (190° C.) in a Dutch oven. Then, the method includes frying the donuts for a time period between about 45 seconds to 1 minute and then flipping and cooking the donuts for a time period of about 45 seconds to 1 minute until the donuts are golden brown in color.

Specific data regarding this example is shown in TABLE 13 and TABLE 14 below.

TABLE 13

| | Raw | Raw | | Raw | Raw | Cooked |
|---|---|---|---|---|---|---|
| | Texture | Density | Raw | Height | MC | Texture |
| Donut | (g) | (g/cm³) | pH | (cm) | (%) | (g) |
| Ova Egg | 123.5 | 1.09 | 5.65 | 3.0 | 34.21 | 198.22 |
| RuBisCO | 114.32 | 1.11 | 5.95 | 3.0 | 30.56 | 163.18 |
| Egg | | | | | | |

TABLE 14

| | Cook | Cooked | Cook | Cook | | | |
|---|---|---|---|---|---|---|---|
| | Water Loss | Density | Height | MC | | | |
| Donut | (%) | (g/cm³) | (cm) | (%) | pH | Color | Sensory |
| Ova Egg | 6.45 | 0.90 | 2.7 | 15.18 | 5.89 | Same | Pass |
| RuBisCO | 5.83 | 0.87 | 3.2 | 14.20 | 6.01 | Same | Pass |
| Egg | | | | | | | |

### Example 25—Pancakes

The components of Example 25 include: approximately 100-200 grams of flour, approximately 10-20 grams of sugar, approximately 5-10 grams of baking powder, approximately 0.5-2 grams of salt, approximately 50-60 grams of butter, approximately 150-250 mL of milk or the whole milk product formulation of Examples 13 or 14, and approximately 25-75 grams of eggs or the egg replacement product formulation of Example 7 or Example 8.

The method to create the pancakes of this example includes mixing the flour, the sugar, the baking powder, and the salt to form a first mixture. Next, the method includes mixing the eggs or the egg replacement product formulation of Example 7 or Example 8, the milk or the whole milk product formulation of Examples 13 or 14, and the butter to form a second mixture. Further, the method includes combining the first mixture with the second mixture to form a third mixture, which is used to form pancake mixture. The pancake mixture is cooked on a medium heat until both sides are golden brown (e.g., about 1-2 minutes per side).

Specific data regarding this example is shown in TABLE 15 and TABLE 16 below.

TABLE 15

| | Raw | Raw | | | Raw | Cooked |
|---|---|---|---|---|---|---|
| | Viscometry | Density | Raw | Raw | MC | Texture |
| Pancakes | (cP) | (g/cm³) | pH | Height | (%) | (g) |
| Ova Egg | 1633 | 0.98 | 6.95 | N/A | 49.93 | 5988.38 |
| RuBisCO | 2250 | 0.94 | 6.89 | N/A | 48.97 | 7826.14 |
| Egg | | | | | | |

TABLE 16

| | Cook | Cooked | Cook | Cook | | | |
|---|---|---|---|---|---|---|---|
| | Water | Density | Height | MC | | | Sen- |
| Pancakes | Loss (%) | (g/cm³) | (cm) | (%) | pH | Color | sory |
| Ova Egg | 13.08 | 0.926 | 1.2 | 35.62% | 7.72 | Same | Pass |
| RuBisCO | 9.90 | 1.09 | 1.4 | 39.38% | 7.31 | Same | Pass |
| Egg | | | | | | | |

### Example 26—Burger Bun

The components of Example 26 include: approximately 350-450 grams of flour, approximately 150-250 grams of warm water, approximately 20-30 grams of butter, approximately 45-65 grams of sugar, approximately 1-5 grams of salt, approximately 5-10 grams of active yeast, and approximately 25-75 grams of eggs or the egg replacement product formulation of Example 7 or Example 8.

The method to create the burger bun of Example 26 includes: mixing all components/ingredients together utilizing an electric stand mixer with a dough hook until the dough is soft and smooth. Next, the method includes covering the dough and allowing the dough to double in size for a time period of 1-2 hours. Then, the method includes gently deflating the dough and dividing the dough into about 8 pieces (˜100 g each). Next, the method includes placing the dough onto a baking sheet, covering the dough, and letting the dough rise for about an hour. Further, the method includes baking the dough at a temperature of 375° F. (190° C.) for a time period of about 15-18 minutes.

Specific data regarding this example is shown in TABLE 17 and TABLE 18 below.

TABLE 17

| | Raw | Raw | | Raw | Raw | Cooked |
|---|---|---|---|---|---|---|
| | Texture | Density | Raw | Height | MC | Texture |
| Burger bun | (g) | (g/cm³) | pH | (cm) | (%) | (g) |
| Ova Egg | 197.88 | 1.02 | 5.38 | 2.9 | 32.01 | 240.06 |
| RuBisCO Egg | 170.14 | 1.12 | 5.57 | 2.8 | 25.34 | 236.2 |

TABLE 18

| | Cook | Cooked | Cook | Cook | | | |
|---|---|---|---|---|---|---|---|
| | Water | Density | Height | MC | | | |
| Burger bun | Loss (%) | (g/cm³) | (cm) | (%) | pH | Color | Sensory |
| Ova Egg | 7.83 | 0.894 | 4.0 | 29.35 | 5.38 | Same | Pass |
| RuBisCO | 9.76 | 0.946 | 3.4 | 21.12 | 5.62 | Same | Pass |
| Egg | | | | | | | |

Example 27—Process for Protein Extraction and Purification to produce Protein Isolates After harvesting and brief washing, plant biomass, for example, from the Lemna genus is lysed in extraction buffer containing 0.1 M NaCl and 2% metabisulfite (without EDTA). Calcium chloride and phosphate (comprising potassium phosphate dibasic and potassium phosphate monobasic) is added to 375 mL of post-basket centrifugation (Rousselet-Robatel Model RA20VxR Vertical Basket Centrifuge; Robatel Inc, Pittsfield, Massachusetts) filtrate.

The filtrate is stirred for 15 minutes at room temperature. After calcium chloride treatment, a 13 mL fraction is taken and spun down on a tabletop centrifuge (Horizon Model 614B Centrifuge; Drucker Diagnostics LLC, Port Matilda, Pennsylvania). Chlorophyll removal can be determined upon analysis of the supernatant and pellet fraction after calcium chloride and phosphate treatment and 5 minutes of centrifugation. Lysate is treated with activated carbon Cabot Norit Americas Inc, Marshall, Texas) (15 minutes) and chitosan (Chitosan (10-120 cps), fungal origin (9012-76-4); Glentham Life Sciences Ltd., Corsham, Wiltshire, UK) (5 minutes) per standard procedure. The activated carbon-chitosan is spun down, and the supernatant is further filtered through 2 coffee filters. Chitosan and activated carbon can function properly after pre-treatment with calcium chloride and phosphate, for example for color removal.

SDS-PAGE Coomassie staining analysis is performed to visualize and determine RuBisCO protein levels in the obtained fractions. Results from the pellet and supernatant (“Sup”) after benchtop centrifugation can show that chlorophyll will still be attached to a protein, or a subunit, size around 25 kDa in the pellet, while the supernatant will contain the majority of the RuBisCO. Calcium chloride with phosphate will selectively precipitate the chlorophyll-binding protein and leave RuBisCO in solution. The results can indicate that calcium chloride efficiently removes chlorophyll and cellular membranes from green filtrate post-basket centrifugation, that the bulk of Rubisco remains in the supernatant, that calcium chloride-induced precipitation appears to occur immediately, and that calcium chloride removes 25 kDa-chlorophyll-binding protein.

### Example 28—Liquid Formulations

To obtain liquid formulations, such as for an egg-white replacement liquid, the RuBisCO compositions can be formulated a set forth in TABLE 19 and 20 below.

TABLE 19

| Ingredient | Mass | Unit | % ABS |
|---|---|---|---|
| Preservative Solution | 88.79 | grams | 88.79% |
| RuBisCO Protein Isolate | 11.21 | grams | 10.96% |
| TOTAL: | 100.00 | grams | 100.00% |

TABLE 20

| Ingredient | Mass | Unit | % ABS |
|---|---|---|---|
| Preservative Solution | 88.79 | grams | 88.79% |
| RuBisCO Protein Isolate | 10.96 | grams | 10.96% |
| Xanthan Gum | 0.25 | grams | 0.25% |
| TOTAL: | 100.00 | grams | 100.00% |

### Example 29—Spray Drying

To obtain dried formulations, such as for an egg-replacement powder, the RuBisCO compositions are formulated a set forth in TABLE 21, TABLE 22, and TABLE 23 below.

TABLE 21

| Ingredient | Mass | Unit | % ABS |
|---|---|---|---|
| Preservative Solution | 71.84 | grams | 71.84% |
| RuBisCO Protein Isolate | 13.38 | grams | 13.38% |
| Safflower Oil | 9.54 | grams | 9.54% |
| Beta Carotene | 0.04 | grams | 0.04% |
| Baking Powder | 4.46 | grams | 4.46% |
| Protein Masker | 0.74 | grams | 0.74% |
| TOTAL: | 100.00 | grams | 100.00% |

TABLE 22

| Ingredient | Mass | Unit | % ABS |
|---|---|---|---|
| Preservative Solution | 70.72 | grams | 70.72% |
| Canola Oil | 9.54 | grams | 9.54% |
| RuBisCO Protein Isolate | 10.90 | grams | 10.90% |
| Beta Carotene | 0.04 | grams | 0.04% |
| Sunflower Lecithin | 0.12 | grams | 0.12% |
| Tapioca Starch | 7.70 | grams | 7.7% |
| Protein Masker | 0.74 | grams | 0.74% |
| Xanthan Gum | 0.24 | grams | 0.24% |
| TOTAL: | 100.00 | grams | 100.00% |

TABLE 23

| Ingredient | Mass | Unit | % ABS |
|---|---|---|---|
| RuBisCO Protein Isolate | 11 | Grams | 11% |
| Preservative Solution | 70.62 | grams | 70.62% |
| Canola Oil | 9.54 | grams | 9.54% |
| Beta Carotene | 0.04 | grams | 0.04% |
| Sunflower Lecithin | 0.12 | grams | 0.12% |
| Tapioca Starch | 7.70 | grams | 7.70% |
| Protein Masker | 0.74 | grams | 0.74% |
| Xanthan Gum | 0.24 | grams | 0.24% |
| TOTAL: | 100.00 | grams | 100.00% |

### Example 29—Preservative Solution

To obtain a preservative solution, such as for such in RuBisCO compositions, such solution are formulated a set forth in TABLE 24 below.

TABLE 24

| Ingredient | Mass | Unit | % ABS |
|---|---|---|---|
| Ascorbic Acid | 0.68 | grams | 0.68% |
| Citric Acid | 0.68 | grams | 0.68% |
| Water | 98.64 | grams | 98.64% |
| Sodium Hydroxide (2M) | | grams | |
| TOTAL: | 100.00 | grams | 100.00% |

The formulations given above can be created using a RuBisCO solution the relevant mass concentration (eliminating a redissolution step), with ultrafiltration used for tuning the concentration of the Rubisco solution. The other components are dissolved into the Rubisco solution using a high shear mixer between 4000-6000 rpm mixing speed for 10-30 minutes depending on batch size. The resulting emulsion is then spray dried under the following conditions: 10-100 L/hour liquid flow rate; 100-170 degrees C. inlet temperature, and 85-95 degrees C. outlet temperature.

### Example 30—RuBisCO Production

Workflow A. One kg of fresh Lemna minor was macerated in a Vitamix Blender (Vitamix Corp, Cleveland, Ohio) in a ratio of 1:1 with a sodium carbonate buffer containing 0.3% w/v sodium bisulfite. The extraction was performed for 3 minutes at medium speed setting maintaining the temperature at less than 30° C. Subsequently, the macerated biomass was filtered by using a nylon straining bag (Natural Home Brands, Sun Valley, California) with a fine mesh to separate the fibrous high solids cake from the liquid juice containing the soluble protein. The filtered homogenate was then centrifuged for 10 minutes at a speed/force of 4000 g (Allegra X15R, SX4750 rotor; Beckman Coulter, Inc., Pasadena, California). The pellet was discarded, and the supernatant was collected separately. The solution was heated to a temperature of 50° C. in a water bath that was set at a temperature of 55° C. and was cooled rapidly to a temperature less than 15° C. after reaching the target temperature. Following the rapid cooling of the protein solution, 2% v/v of activated chitosan and 4% w/v of activated carbon (Cabot Norit Americas Inc, Marshall, Texas) is added to the liquid juice. The solution was subsequently stirred for 5 minutes after which the solution was centrifuged for 10 minutes at a speed/force of 5000 g (Allegra X15R, SX4750 rotor; Beckman Coulter, Inc., Pasadena, California). The green pellet in the centrifuge bottle was discarded, and the clear yellow supernatant was micro filtered using a 0.7 pm Glass Micro fiber membrane (Whatman 1825-047 Glass Microfiber Binder Free Filter, 0.7 Micron; Global Life Sciences Solutions USA LLC, Marlborough, Massachusetts). The filtrate was subsequently exposed to a 0.2 pm polyethersulfone membrane (Polyethersulfone (PES) Membrane Filters, 0.2 Micron; Sterlitech Corporation Inc, Kent, Washington) to remove the remainder of the undesired particles including bacteria. The obtained pale yellow and deodorized proteinaceous solution was then concentrated using a 70 kDa membrane (MINIKROS® S02-E070-05-N; Spectrum Laboratories, Inc., Rancho Dominguez, California). The concentrated solution obtained was subsequently freeze dried (Harvest Right LLC, Salt Lake City, Utah) and the result was a white, odorless and soluble protein powder.

Workflow B. One kg of fresh Lemna minor was macerated using a Vitamix Blender (Vitamix Corp, Cleveland, Ohio) in a ratio of 1:1 with a potassium phosphate buffer containing 0.3% w/v ascorbic acid. The maceration was performed for a period of 3 minutes at medium speed in order to maintain a temperature of less than 30° C. The lysed biomass was filtered by using a nylon straining bag (Natural Home Brands, Sun Valley, California) with a fine mesh to separate the fibrous high solids cake from the liquid juice containing the soluble protein. The filtered homogenate was then centrifuged for 10 minutes at a speed/force of 4000 g (Allegra X15R, SX4750 rotor; Beckman Coulter, Inc., Pasadena, California). The pellet was discarded, and the supernatant was collected separately. The supernatant was then mixed with 5% v/v of activated chitosan (Chitosan (10-120 cps), fungal origin (9012-76-4); Glentham Life Sciences Ltd., Corsham, Wiltshire, UK) and 10% w/v of activated carbon (Cabot Norit Americas Inc, Marshall, Texas) for a period of 5 minutes. Subsequently the mixed solution was centrifuged at a speed/force of 5000 g for 10 minutes (Allegra X15R, SX4750 rotor; Beckman Coulter, Inc., Pasadena, California). The obtained pellet was discarded, and the deodorized and decolored supernatant was microfiltered using a 0.2 pm polyethersulfone membrane (Polyethersulfone (PES) Membrane Lilters, 0.2 Micron; Sterlitech Corporation Inc, Kent, Washington). The obtained pale yellow and deodorized proteinaceous solution was then concentrated using a 70 kDa membrane (MINIKROS® 502-E070-05-N; Spectrum Laboratories, Inc., Rancho Dominguez, California). The concentrated solution obtained was subsequently freeze dried (Harvest Right LLC, Salt Lake City, Utah) and the result was a white, odorless and soluble protein powder. Workflow C. One kg of fresh Lemna minor was macerated using a Vitamix Blender (Vitamix Corp, Cleveland, Ohio) in a ratio of 1:1 with distilled water containing 0.3% w/v of sodium bisulfite and ascorbic acid. The maceration was performed for a period of 3 minutes at medium speed in order to maintain a temperature of less than 30° C. The lysed biomass was filtered by using a nylon straining bag (Natural Home Brands, Sun Valley, California) with a fine mesh to separate the fibrous high solids cake from the liquid juice containing the soluble protein. The filtered homogenate was then centrifuged for 10 minutes at a speed/force of 4000 g. The pellet was discarded, and the supernatant was collected separately. The supernatant was then mixed with a solution containing 30 mM of potassium phosphate and 20 mM of calcium chloride for a period of 5 minutes. Subsequently the mixed solution was centrifuged at a speed/force of 5000 g for 10 minutes (Allegra X15R, SX4750 rotor; Beckman Coulter, Inc., Pasadena, California). The obtained pellet was discarded. 5% w/v of activated carbon (Cabot Norit Americas Inc, Marshall, Texas) was added to the supernatant, and the solution was stirred for 5 minutes. Subsequently, the mixed solution containing the activated carbon was micro filtered using a 0.2 pm polyethersulfone membrane filter (Polyethersulfone (PES) Membrane Filters, 0.2 Micron; Sterlitech Corporation Inc, Kent, Washington) in order to remove the activated carbon that had adsorbed the remaining chlorophyll, polyphenol and other unwanted taste/color/odor impacting particles. The obtained pale yellow and deodorized proteinaceous solution was then concentrated using a 100 kDa membrane (Hollow Fiber Cartridge, 100,000 NMWC, 850 cm2; GE Healthcare Bio-Sciences Corp, Westborough, Massachusetts). The concentrated solution obtained was subsequently freeze dried and the result was a white, odorless and soluble protein powder.

Workflow D. One kg of fresh Lemna minor was macerated using a Vitamix Blender (Vitamix Corp, Cleveland, Ohio) in a ratio of 1:1 with distilled water containing 0.5% w/v of sodium bisulfite. The maceration was performed for a period of 3 minutes at medium speed in order to maintain a temperature of less than 30° C. The lysed biomass was filtered by using a nylon straining bag (Natural Home Brands, Sun Valley, California) with a fine mesh to separate the fibrous high solids cake from the liquid juice containing the soluble protein. The filtered homogenate was then centrifuged for 10 minutes at a speed/force of 4000 g (Allegra X15R, SX4750 rotor; Beckman Coulter, Inc., Pasadena, California). The pellet was discarded, and the supernatant was collected separately. The supernatant was then mixed with a solution containing 30 mM of potassium phosphate and 20 mM of calcium chloride for a period of 5 minutes. Subsequently the mixed solution was centrifuged at a speed/force of 5000 g for 10 minutes (Allegra X15R, SX4750 rotor; Beckman Coulter, Inc., Pasadena, California). The obtained pellet was discarded. 2% w/v of activated chitosan (Chitosan (10-120 cps), fungal origin (9012-76-4); Glentham Life Sciences Ltd., Corsham, Wiltshire, UK) and 4% of activated carbon (Cabot Norit Americas Inc, Marshall, Texas) were added to the supernatant, and the solution was stirred for 5 minutes. Subsequently the mixed solution was centrifuged at a speed/force of 5000 g for 10 minutes (Allegra X15R, SX4750 rotor; Beckman Coulter, Inc., Pasadena, California). The obtained pellet was discarded, and the deodorized and decolored supernatant was microfiltered using a 0.7 pm polyethersulfone membrane (Whatman 1825-047 Glass Microfiber Binder Free Filter, 0.7 Micron; Global Life Sciences Solutions USA LLC, Marlborough, Massachusetts). The filtrate was then further microfiltered using a 0.2 pm polyethersulfone membrane (Polyethersulfone (PES) Membrane Filters, 0.2 Micron; Sterlitech Corporation Inc, Kent, Washington). The obtained pale yellow and deodorized proteinaceous solution was then concentrated using a 70 kDa membrane (MINIKROS® 502-E070-05-N; Spectrum Laboratories, Inc., Rancho Dominguez, California). The concentrated solution obtained was subsequently freeze dried (Harvest Right LLC, Salt Lake City, Utah) and the result was a white, odorless and soluble protein powder.

Purity analysis from Workflows A-D. The average purity of the protein preparations prepared by the methods of Workflows A-D was about 84.3% and the concentration of soluble protein after ultrafiltration was 1,316 pg/mL. The foaming capacity achieved was 195% and maintained a 92% stability after 1 hour. Gelation properties of the freeze-dried material were validated, and only 2% w/v of freeze-dried material was needed to be added in order to form a gel.

Comparative Analysis. Lemna leaf proteins were extracted as described in WO2011/0778671 A1 (van de Velde et al.) with some modifications. One kg of fresh Lemna was washed and macerated using a Vitamix Blender at a ratio of 2:1 with 0.3% w/v sodium bisulfite. The homogenate was sieved through a cheese cloth prior to heating up to 60° C. The filtrate was kept at 60° C. for 5 minutes and then cooled down to 10° C. Following the heat treatment, the suspension was centrifuged for 5 minutes at 5200 g. Next, activated carbon was added to the supernatant in an amount of 5% w/w. Following the addition of the activated carbon, the suspension was stirred for 5 minutes before the supernatant was removed by decanting.

The supernatant obtained was subjected to two microfiltration steps. First, the supernatant was passed over a microfilter having a pore size of 0.7 pm (Whatman 1825-047 Glass Microfiber Binder Free Filter, 0.7 Micron; Global Life Sciences Solutions USA LLC, Marlborough, Massachusetts). Subsequently, the filtrate was passed over a microfilter having a pore size 0.2 pm (Polyethersulfone (PES) Membrane Filters, 0.2 Micron; Sterlitech Corporation Inc, Kent, Washington). The filtrate was then freeze dried and the result was a whitish and odorless powder.

The purity of the protein was approximately 34.1% per unit of dry matter and the concentration of soluble protein prior to freeze-drying was 520 pg/mL. Purity of Examples 1˜4 and Comparative Example 1 are shown in TABLE 25. The foaming properties of the freeze-dried material showed a total foaming strength of 92% with a stability of 62% after 1 hour. Gelation properties of the freeze-dried material were validated, and at least 7% w/v of freeze-dried material was needed to be added in order to form a gel.

TABLE 25

| Sample | Purity (% per unit of dry matter) |
|---|---|
| Example 1 | 88.2 |
| Example 2 | 85.2 |
| Example 3 | 82.1 |
| Example 4 | 78.9 |
| Comparative Example 1 | 34.1 |

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A composition, wherein the composition is in a dried form, and wherein the composition comprises:
a ribulose-1,5-bisphosphate carboxylase/oxygenase (RuBisCO) protein isolate;
a plasticizer, wherein a ratio by weight of the plasticizer to the RuBisCO protein isolate is about 1:5 to 1:200; and
one or more food additives.

2. The composition of claim 1, wherein a ratio by weight of the plasticizer to the RuBisCO protein isolate is about 1:5 to 1:10.

3. The composition of claim 1, wherein the plasticizer comprises a gum.

4. The composition of claim 3, wherein the gum comprises xanthan gum, acacia gum, gellan gum, guar gum, locust bean gum, tragacanth gum, carrageenan gum, or a combination thereof.

5. The composition of claim 3, wherein a ratio by weight of the gum to the RuBisCO protein isolate is about 1:40 to 1:50.

6. The composition of claim 3, wherein the gum is xanthan gum.

7. The composition of claim 1, wherein the RuBisCO protein isolate is free of chlorophyll, is flavorless, is colorless, or any combination thereof.

8. The composition of claim 1, wherein the RuBisCO protein isolate comprises a RuBisCO protein large subunit and/or a RuBisCO protein small subunit.

9. The composition of claim 1, wherein the RuBisCO protein isolate comprises protein comprising a sequence at least 90% identical, or optionally at least 95% identical, to the sequences set forth in any one of SEQ ID NOS: 1-10.

10. The composition of claim 1, wherein the RuBisCO protein isolate comprises protein comprising a sequence of SEQ ID NO: 1 and 2; SEQ ID NO: 3 and 4; SEQ ID NO: 5 and 6; SEQ ID NO: 7 and 8; or SEQ ID NO: 9 and 10.

11. The composition of claim 1, wherein the RuBisCO protein isolate is from a plant in the Lemna genus.

12. The composition of claim 11, wherein the RuBisCO protein isolate is from a Lemna minor.

13. The composition of claim 1, wherein the RuBisCO protein isolate is from a Lemna aequinoctialis, Lemna disperma, Lemna ecuadoriensis, Lemna gibba, Lemna japonica, Lemna minor, Lemna minuta, Lemna obscura, Lemna paucicostata, Lemna perpusilla, Lemna tenera, Lemna trisulca, Lemna turionifera, Lemna valdiviana, Lemna yungensis, Nicotiana sylvestris, Nicotiana tabacum, Medicago sativa, Spinacia oleracea, Beta vulgaris, Atriplex lentiformis, Pereskia aculeata, or Chlorella vulgaris.

14. The composition of claim 1, wherein the one or more food additive is one or more of: a sugar, a flavoring component, a coloring component, a fiber, a soluble salt, a starch, an acid, and a wax.

15. The composition of claim 14, wherein the flavoring component comprises one or more of salt, spices, turmeric, cinnamon, cloves, allspice, ginger, vanilla, vanilla extract, vanilla flavoring, sugar, tartar, sweeteners, monosodium glutamate, chocolate chips, coco powder, nuts, sulfuric flavoring components, and a flavor masker.

16. The composition of claim 14, wherein the fiber is selected from the group consisting of: bran, psyllium fiber, citrus fiber, bamboo fiber, carrot fiber, oat fiber, cellulose, methylcellulose, crystalline 5 cellulose, pectin, or any combination thereof.

17. The composition of claim 14, wherein the starch comprises one or more of arrowroot starch, cornstarch, tapioca starch, mung bean starch, potato starch, sweet potato starch, rice starch, sago starch, and wheat starch.

18. The composition of claim 14, wherein the soluble salt is calcium lactate gluconate.

19. The composition of claim 14, wherein the acid is selected from the group consisting of: ascorbic acid and citric acid.

20. The composition of claim 14, wherein the wax is selected from the group consisting of: a naturally-derived wax and a synthetic wax.

21. The composition of claim 1, wherein the composition further comprises lecithin.

22. The composition of claim 1, further comprising one or more of an animal ingredient, a plant-based ingredient, or a synthetic ingredient.

23. The composition of claim 22, wherein the animal ingredient comprises one or more of an egg, dairy, and meat from an animal.

24. The composition of claim 22, wherein the plant-based ingredient comprises one or more of a vegetable, fruit, root, or nut.

25. The composition of claim 22, wherein the synthetic ingredient comprises one or more of artificial flavorings.

26. The composition of claim 1, wherein the RuBisCO protein isolate comprises up to 20% by weight of the composition.

27. The composition of claim 1, wherein the RuBisCO protein isolate comprises more than 30% by weight of the composition.

28. The composition of claim 1, wherein each food additive is present in amount of up to about 10% by weight.


SEQUENCE LISTING (10 sequences)

SEQ ID NO: 1 - protein, 478 aa, Lemna minor
MSPQTETKASAGFKAGVKDYKLNYYTPEYETKDTDILAAFRVTPQPGVPPEEAGAAVAAESSTGTWTTVWTDGLTSLDRYKGRCYHIEPVAGEENQFIAYIAYPLDLFEEGSVTNMFTSIVGNVFGFKALRALRLEDLRIPPAYSKTFQGPPHGIQVERDKLNKYGRPLLGCTIKPKLGLSAKNYGRAVYECLRGGLDFTKDDENVNSQPFMRWRDRFLFCAEAIYKAQAETGEIKGHYLNATAGTCEEMIKRAVFARELGVPIVMHDYLTGGFTANTSLAYYCRDNGLLLHIHRAMHAVIDRQKNHGMHFRVLAKALRMSGGDHVHSGTVVGKLEGEREMTLGFVDLLRDDFIEKDRSRGIFFTQDWVSMPGVLPVASGGIHVWHMPALTEIFGDDSVLQFGGGTLGHPWGNAPGAVANRVALEACVKARNEGRDLAREGNEIIREACNWSPELAAACEVWKEIKFEYEPVDKLDVK

SEQ ID NO: 2 - protein, 177 aa, Lemna minor
MASSMMASTAAAVARAGPAQSSMVPFNACRSSVPFPATRKANNNLSTLPGNGGRVSCMQVWPPEGLKKFETLSYLPPLSVEDLAKEVDYLLRNDWVPCIEFSKEGFVYRENHASPGYYDGRYWTMWKLPMFGCTDASQVIAEVEEAKKAYPEYFVRIIGFDNKRQVQCISFIAYKPT

SEQ ID NO: 3 - protein, 477 aa, Nicotiana tabacum
MSPQTETKASVGFKAGVKEYKLTYYTPEYQTKDTDILAAFRVTPQPGVPPEEAGAAVAAESSTGTWTTVWTDGLTSLDRYKGRCYRIERVVGEKDQYIAYVAYPLDLFEEGSVTNMFTSIVGNVFGFKALRALRLEDLRIPPAYVKTFQGPPHGIQVERDKLNKYGRPLLGCTIKPKLGLSAKNYGRAVYECLRGGLDFTKDDENVNSQPFMRWRDRFLFCAEALYKAQAETGEIKGHYLNATAGTCEEMIKRAVFARELGVPIVMHDYLTGGFTANTSLAHYCRDNGLLLHIHRAMHAVIDRQKNHGIHFRVLAKALRMSGGDHIHSGTVVGKLEGERDITLGFVDLLRDDFVEQDRSRGIYFTQDWVSLPGVLPVASGGIHVWHMPALTEIFGDDSVLQFGGGTLGHPWGNAPGAVANRVALEACVKARNEGRDLAQEGNEIIREACKWSPELAAACEVWKEIVFNFAAVDVLDK

SEQ ID NO: 4 - protein, 181 aa, Nicotiana tabacum
MAFLIMSSAAAVATGTNAAQASMIAPFTGLKSATSFPVSRKQNLDITSIASNGGRVQCMQVWPPINKKKYETLSYLPDLSEEQLLREVEYLLKNGWVPCLEFETEHGFVYRENNKSPGYYDGRYWTMWKLPMFGCTDATQVLAEVEEAKKAYPQAWIRIIGFDNVRQVQCISFIAYKPEGY

SEQ ID NO: 5 - protein, 474 aa, Medicago sativa
MSPQTETKATVGFKAGVKDYRLTYYTPDYETKDTDILAAFRVSPQPGVPAEEAGAAVAAESSTGTWTTVWTDGLTSLDRYKGRCYHIEPVAGEETQFIAYVAYPLDLFEEGSVNYMFTSIVGNVFGFKALRALRLEDLRIPAAYVKTFQGPPQGIQVERDKLNKYGRPLLGCTIKPKLGLSAKNYGRAVYECLRGGLDFTKDDENVNSQPFMRWRDRFLFCAEAIYKAQAETGEIKGHYLNATAGTCEEMMKRAVFARELGVPIVMHDYLTVGFTANTTLAHYCRDNGLLLHIHRAMHAVIDRQKNHGMHFRVLAKALRMSGGDHIHAGTVVGKLEGERDITLGFVDLLRDDFIEKDRSRGIFFTQDWVSLPGVLPVASGGIHVWHMPALTEIFGDDSVLQFGGGTLGHPWGNAPGAVANRVALEACVQARNEGRDLAREGNEIIREATKWSPELAAACEVWKEIKFEFPAMDN

SEQ ID NO: 6 - protein, 180 aa, Medicago sativa
MALISSAAVTTVNRASSAQANLVAPFTGLKSSAGFPVTKKTNNDITSIASNGGRVNCMQVWPPVGKKKFETLSYLPPLTEEQLAKEVEYLIRKGWIPCLEFELEKGFVYRENHRSPGYYDGRYWTMWRLPLFGATDSSQVLKELADCKAEYPDSFIRIIGFDNVRQVQCISFIAHTPKNY

SEQ ID NO: 7 - protein, 475 aa, Spinacia oleracea
MSPQTETKASVEFKAGVKDYKLTYYTPEYETLDTDILAAFRVSPQPGVPPEEAGAAVAAESSTGTWTTVWTDGLTNLDRYKGRCYHIEPVAGEENQYICYVAYPLDLFEEGSVTNMFTSIVGNVFGFKALRALRLEDLRIPVAYVKTFQGPPHGIQVERDKLNKYGRPLLGCTIKPKLGLSAKNYGRAVYECLRGGLDFTKDDENVNSQPFMRWRDRFLFCAEALYKAQAETGEIKGHYLNATAGTCEDMMKRAVFARELGVPIVMHDYLTGGFTANTTLSHYCRDNGLLLHIHRAMHAVIDRQKNHGMHFRVLAKALRLSGGDHIHSGTVVGKLEGERDITLGFVDLLRDDYTEKDRSRGIYFTQSWVSTPGVLPVASGGIHVWHMPALTEIFGDDSVLQFGGGTLGHPWGNAPGAVANRVALEACVQARNEGRDLAREGNTIIREATKWSPELAAACEVWKEIKFEFPAMDTV

SEQ ID NO: 8 - protein, 123 aa, Spinacia oleracea
MQVWPPLGLKKFETLSYLPPLTTEQLLAEVNYLLVKGWIPPLEFEVKDGFVYREHDKSPGYYDGRYWTMWKLPMFGGTDPAQVVNEVEEVKKAPPDAFVRFIGFNDKREVQCISFIAYKPAGY

SEQ ID NO: 9 - protein, 475 aa, Chlorella vulgaris
MSPQTETKARVGFKAGVKDYRLTYYTPDYQPKDTDILAAFRMTPQPGVPPEEAGAAVAAESSTGTWTTVWTDGLTSLDRYKGRCYDIEPVPGEENQYIAYIAYPLDLFEEGSVTNLFTSIVGNVFGFKALRALRLEDLRIPPAYVKTFQGPPHGIQVERDKLNKYGRGLLGCTIKPKLGLSAKNYGRAVYECLRGGLDFTKDDENVNSQPFMRWRDRFLFVAEAIYKSQAETGEIKGHYLNATAATAEAMMQRAECAKDLGVPIIMHDYLTGGFTANTSLSHYCRDNGLLLHIHRAMHAVIDRQRNHGITFRVLAKALRLSGGDHLHSGTVVGKLEGEREVTLGFVDLMRDDYIEKDRSRGIYFTQDWVSLPGTMPVASGGIHVWHMPALVEIFGDDACLQFGGGTLGHPWGNAPGAAANRVALEACTQARNEGRDLAREGGDVIRAACKWSPELAAACEVWKEIKFEFETIDTL

SEQ ID NO: 10 - protein, 214 aa, Chlorella vulgaris
MAALTASLVSCPVAVAAKPARLASPAWPASLSPRRLSPPSPRGPSQTAAAPARCSCGSPLTTSSSRPSPTCPSDRRPDRQAGRLHHPSGWTPALEFSNAESAYVKDVANIRFTGGSASCNYYDNRYWAMYKLPMFGCTDASQVLAEIANAVKTFPDSYVRMAAFDAVRQVQTVAILVHRPASATDYRLPENPQPLIGCTTQLERPQYCKSLANL